(19) United States
(12) Patent Application Publication
(10) Pub. No.: US 2026/0162255 A1
(43) Pub. Date: Jun. 11, 2026

(54) SYSTEMS AND METHODS FOR IDENTIFICATION OF RARE EVENTS IN BIOLOGICAL SAMPLES

(71) Applicant: University of Southern California, Los Angeles, CA (US)

(72) Inventors: Peter KUHN, Solana Beach, CA (US); Assad OBERAI, Los Angeles, CA (US); Javier MURGOITIO ESANDI, Los Angeles, CA (US)

(73) Assignee: University of Southern California, Los Angeles, CA (US)

(21) Appl. No.: 19/380,408

(22) Filed: Nov. 5, 2025

Related U.S. Application Data
(60) Provisional application No. 63/716,845, filed on Nov. 6, 2024.

(51) Int. Cl. G06T 7/00; A61B 10/00; G01N 21/64; G01N 33/487; G06N 3/0455; G06V 10/764; G06V 10/774; G06V 10/82; G06V 20/69

(52) U.S. Cl. CPC G06T 7/0012; A61B 10/0045; G01N 21/6428; G01N 21/6456; G01N 33/487; G06N 3/0455; G01N 2021/6439; G06T 2207/10056; G06T 2207/10064; G06T 2207/20021; G06T 2207/20081; G06T 2207/20084; G06T 2207/30024; G06T 2207/30204; G06V 10/764; G06V 10/774; G06V 10/82; G06V 20/698

(57) ABSTRACT

This disclosure relates to a liquid biopsy system for identifying rare events to determine a subject's disease state and propose treatment strategies. The system generates or obtains an image of a biological sample, divides the image into multiple tiles, and processes tile data using an autoencoder (e.g., Wasserstein). The autoencoder is trained to reproduce each tile, and a reproducibility difference is calculated for each. Tiles are ranked based on their reproducibility differences, enabling identification of rare cellular or molecular events. These rankings support disease state determination and inform treatment strategy selection.

### CROSS-REFERENCE TO RELATED APPLICATIONS

This application claims the benefit of U.S. provisional application Ser. No. 63/716,845 filed Nov. 6, 2024, the disclosure of which is hereby incorporated in its entirety by reference herein.

### TECHNICAL FIELD

This disclosure relates to a liquid biopsy system. This disclosure also relates to a rare event identification system and method for determining a subject's disease state.

### BACKGROUND

Typical approaches applied to detect rare cells by using immunofluorescence (IF) microscopy may rely on the following three steps: (1) All cells are segmented on a given immunofluorescence (IF) image where each IF-image can contain a few million cells, and wherein each cell is identified and delineated from the background to obtain segmented cell image, (2) then, cell features, including, for example, cell and nuclear area, nuclear eccentricity, etc., together with statistics of IF intensities of each immunofluorescence channel, are extracted from each segmented cell image, which may be achieved by processing each segmented cell image, and (3) finally, cells that are outliers in the space of cell features are identified, wherein such outlier cells have features that are very different from most other cells, which is achieved using approaches like k-means clustering.

### RELATED ART REFERENCES

The following publications are related art for the background of this disclosure. One-digit or two-digit numbers in the parentheses before each reference correspond to the numbers in parentheses used in the other parts of this disclosure. The entire content of each reference, including its supplemental content, if available, is incorporated herein by reference.
(1) Ilya Tolstikhin, Olivier Bousquet, Sylvain Gelly, Bernhard Scholkopf “Wasserstein auto-encoders.” Published in International Conference on Learning Representations, 5 Nov. 2017. arXiv preprint arXiv: 1711.01558 (2017).
(2) Huang, Gao, et al. “Densely connected convolutional networks.” Proceedings of the IEEE conference on computer vision and pattern recognition. 2017.
(3) Setayesh, S. M., Hart, O., Naghdloo, A., Higa, N., Nieva, J., Lu, J., and Kuhn, P. (2022). Multianalyte liquid biopsy to aid the diagnostic workup of breast cancer. NPJ Breast Cancer, 8 (1), 112.

### SUMMARY

This disclosure relates to a rare event identification system. This disclosure also relates to a rare event identification system and method that can be used to determine a subject's disease state.

This invention disclosure describes a non-parametric approach for detecting biologically relevant rare events, wherein this approach can circumvent some of the components of previous systems and methods that are either time-consuming or require human intervention.

In this disclosure, the rare event identification system may be configured to generate/obtain an image of a (e.g., liquid) sample; section each sample image into a plurality of tiles; initiate an (e.g., Wasserstein) autoencoder to process data related to tiles; use data related to a number or all tiles to train the autoencoder such that the autoencoder learns to reproduce each tile; determine a reproducibility difference for each tile; and rank each tile according to the reproducibility difference of each tile.

In this disclosure, the rare event identification system may be configured to initiate an autoencoder to process data related to a plurality of tiles; use data related to a number or all tiles to train the autoencoder such that the autoencoder learns to reproduce each tile; determine a reproducibility difference for each tile; and rank each tile according to the reproducibility difference of each tile.

In this disclosure, the rare event identification system may be configured to obtain an image of a sample; section each sample image into a plurality of tiles; provide a trained autoencoder; initiate the trained autoencoder to process data related to the tiles; determine a reproducibility difference for each tile; and rank each tile according to the reproducibility difference of each tile.

In this disclosure, the rare event identification system may be configured to provide a trained autoencoder; initiate the trained autoencoder to process data related to a plurality of tiles; determine a reproducibility difference for each tile; and rank each tile according to the reproducibility difference of each tile.

The image of the sample is hereafter referred to as the sample image. The sample image includes a plurality of pixels and the intensity of each of said pixels. The tile is a sectioned image of the sample image. The sample image is sectioned into a sufficient number of tiles to train the autoencoder. The tile's data that is input to the autoencoder is hereafter referred to as the original tile's data. The tile's output data from the autoencoder is hereafter referred to as the reproduced tile's data. The reproducibility difference is the difference between each original tile's data and a reproduced tile's data.

In this disclosure, the rare event identification system may be configured to initiate an (e.g., Wasserstein) autoencoder to process data related to tiles; use data related to a number or all tiles to train the autoencoder such that the autoencoder learns to reproduce each tile; determine a reproducibility difference for each tile; and rank each tile according to the reproducibility difference of each tile.

In this disclosure, the rare event identification system may further be configured to identify rare tiles that may not be relevant to the object from which the sample is obtained. The non-relevant tiles may contain events that may be erroneously introduced into the sample or the sample image or the tile image during the obtaining or processing the sample image or the tile image. In this disclosure, said non-relevant tiles are referred to as biologically non-relevant tiles. For example, the biologically non-relevant tiles may contain events that may be introduced as artifacts and/or due to sampling errors and/or sample processing errors and/or system errors.

In this disclosure, the rare event identification system may be configured to generate/obtain an image of a (e.g., liquid) sample; section each sample image into a plurality of tiles; initiate an (e.g., Wasserstein) autoencoder to process data related to tiles; use data related to a number or all tiles to train the autoencoder such that the autoencoder learns to reproduce each tile; determine a reproducibility difference for each tile; and rank each tile according to the reproducibility difference of each tile. This exemplary system is further configured to remove biologically non-relevant events.

In this disclosure, the rare event identification system may be configured to generate/obtain an image of a (e.g., liquid) sample; section each image into image frames; section each sample image into a plurality of tiles; initiate an (e.g., Wasserstein) autoencoder to process data related to tiles; use data related to a number or all tiles to train the autoencoder such that the autoencoder learns to reproduce each tile; determine a reproducibility difference for each tile; rank each tile according to the reproducibility difference of each tile; map back each rare tile back to its spatial location on the sample image; count the number of rare tiles on each image frame; and remove rare tiles from said image frame if a number of said rare tiles present in one image frame exceeds a predetermined threshold rare tile number. The threshold rare tile number may be at least ten rare tiles, or 100 rare tiles, or 1,000 rare tiles in one image frame said tiles belong. The size of each image frame is larger than that of a tile. For example, the number of tiles in one frame may be in a range of 100 to 1,000, or a range of 100 to 10,000, or a range of 100-100,000, or a range of 500 to 5,000. For example, the number of tiles in one frame may be in the range of 500 to 5,000. In this example, the sample may be derived (e.g., obtained) from an object, such as a human subject.

In this disclosure, the rare event identification system may be configured to generate/obtain an image of a (e.g., liquid) sample; section each sample image into a plurality of tiles; initiate an (e.g., Wasserstein) autoencoder to process data related to tiles; use data related to a number or all tiles to train the autoencoder such that the autoencoder learns to reproduce each tile; determine a reproducibility difference for each tile; rank each tile according to the reproducibility difference of each tile; use a trained convolutional neural network (CNN); and remove biologically non-relevant tiles. In this example, the sample may be derived (e.g., obtained) from an object, such as a human subject.

In this disclosure, the trained CNN may be obtained by training a CNN on two sets of training tiles. The first training tile set may include images of biologically non-relevant events, and the second training tile set may include only images of relevant events. The CNN may be trained by obtaining a sample image sectioned into a plurality of tiles, each tile containing data representing a portion of the sample image; creating a first set of training tiles comprising images of biologically non-relevant events; creating a second set of training tiles comprising images of biologically relevant events; training the CNN on the first and second sets of training tiles to classify each tile as either biologically relevant or biologically non-relevant based on its learned features; and thereby obtaining a trained CNN. The CNN may be trained to classify tiles based on features extracted from intensity values across multiple channels of the sample image, wherein each channel is associated with a specific biomarker. The rare event identification system may be further configured to apply the trained CNN to a new set of tiles obtained from a sample image and to remove tiles classified as biologically non-relevant, thereby isolating tiles with biologically relevant events. The rare event identification system may be further configured to train a CNN on a first set of training tiles and a second set of training tiles to obtain a trained CNN, wherein the first set of training tiles comprises tiles with biologically non-relevant events; and wherein the second set of training tiles comprises tiles with biologically relevant events; and apply the trained CNN to remove tiles classified as biologically non-relevant, thereby isolating tiles with biologically relevant events for further analysis. The CNN may be trained with a supervised learning approach using a loss function that minimizes classification error between biologically relevant and non-relevant tiles.

In this disclosure, the trained autoencoder may be obtained by training an autoencoder using an unsupervised approach, a semi-supervised approach, or a supervised learning approach.

In this disclosure, the trained CNN may be obtained by training a CNN using an unsupervised approach, a semi-supervised approach, or a supervised learning approach.

In this disclosure, the first set of training tiles and the second set of training tiles are created by an unsupervised approach, a semi-supervised approach, or a supervised learning approach.

This invention also relates to a method for determining a disease (e.g., cancer) state of a subject and/or proposing a treatment strategy for the subject. This method may use any rare event identification system of this disclosure to achieve these purposes. According to an exemplary method, a thin layer of a liquid sample is first formed on a flat substrate. Then, this liquid sample is stained using an immunofluorescence antibody. The stained liquid sample is scanned using an optical system of this disclosure. This scanning generates an image of the liquid sample. Then, this sample image is sectioned into a plurality of tiles. The rare event detection initiates an (e.g., Wasserstein) autoencoder to process data related to tiles; uses data related to a number or all tiles to train the autoencoder such that the autoencoder learns to reproduce each tile; determines a reproducibility difference for each tile; ranks each tile according to the reproducibility difference of each tile; and remove biologically non-relevant events. This exemplary method is further configured to determine a cancer state for the subject and propose a treatment strategy.

In this disclosure, the autoencoder may be any autoencoder. For example, the autoencoder may be a Wasserstein autoencoder (WAE), a denoising autoencoder, a sparse autoencoder, a deep autoencoder, a contractive autoencoder, an under-complete autoencoder, a convolutional autoencoder, a variational autoencoder, or a combination thereof. For example, the autoencoder may be a Wasserstein autoencoder (WAE).

In this disclosure, each tile may include a partial or complete image of at least one event. For example, the number of events in each tile may be in a range of 1 to 5, or a range of 1 to 10, or a range of 1 to 20, or a range of 1 to 30, or a range of 1 to 50, or a range of 1 to one 100, or a range of 1 to 1,000. For example, the number of events in each tile may be in a range of 1 to 5, or a range of 1 to 10.

In this disclosure, the sample image may be sectioned into at least 100 tiles, or at least 1,000 tiles, or at least 10,000 tiles, or at least 10,000,000 tiles, or at least 100,000,000 tiles. The maximum number of tiles to which the sample image is sectioned may be less than or equal to the total number of pixels of the sample image. For example, each tile may have a size of at least 100 pixels, or at least 1,000 pixels, or at least 10,000 pixels, or at least 50,000 pixels, or at least 100,000 pixels, or at least 1,000,000 pixels. For example, each tile may have a size of at least 100 pixels, or at least 1,000 pixels, or at least 10,000 pixels. For example, each tile may have a size in a range of 100 pixels to 1,000 pixels, or a range of 100 pixels to 10,000 pixels, or a range of 100 pixels to 100,000 pixels, or a range of 1,000 pixels to 10,000 pixels. For example, each tile may have a size such that it can contain at least one partial or complete image of at least one event. For example, each tile may have a size such that it can include at least one event but no more than 3, or no more than 5, or no more than 10, or no more than 30, or no more than 50, or no more than 100, or no more than 1,000 partial or complete images of events. For example, each tile may have a size such that it can contain at least one event but at most 3, or at most 5 partial or complete images of events.

In this disclosure, the reproducibility difference may be determined, for example, by using calculated values of a weighted vector norm, a weighted tensor norm, a vector norm, a tensor norm, or a combination thereof of the difference between the original tile's data and the reproduced tile's data; and wherein a calculated value of said norms or a combination thereof is hereafter referred to as rarity metric. For example, the reproducibility difference may be determined using calculated values of a vector norm or a tensor norm of the difference between the original tile's data and the reproduced tile's data. For example, the reproducibility difference is determined by using calculated values of a vector norm of the difference between the original tile's data and the reproduced tile's data, wherein the vector norm is an LP norm defined by Equation (2), or L^{∞} norm defined by Equation (3), or a combination thereof. For example, the reproducibility difference may be determined by using calculated values a vector norm or a tensor norm, wherein the vector norm is an L¹ norm or an L² norm, or an L³ norm, or an L⁴ norm, or an L^{∞} (L-infinity) norm, or a combination thereof. For example, the reproducibility difference may be determined using calculated values, a vector norm, or a tensor norm, wherein the vector norm is an L¹ norm, an L² norm, or a combination thereof.

In this disclosure, the tiles may be ranked from most rare to least rare or normal, according to the rarity metric of each tile. Or the tiles may be ranked from least rare or normal to most rare, according to the rarity metric of each tile. A tile referred to as a rare tile may have a high rarity metric value. A tile that is referred to as a normal tile or a least rare tile may have a low rarity metric value. A tile referred to as a rarest tile may have the highest rarity metric value. A tile referred to as a most normal tile may have the smallest rarity metric value. For example, a rare tile may be determined using a value of the calculated vector norm or tensor norm and a threshold rarity metric value for the computed norm's value or the calculated tensor's value. For example, the threshold rarity metric may be predetermined such that the rare event identification system may identify in a range of 1 to 10 rare tiles, or a range of 1 to 100 rare tiles, or a range of 1 to 1,000 rare tiles, or a range of 1 to 10,000 rare tiles, or a range of 1 to 100,000 rare tiles, which have rarity index values larger than those of remaining rare tiles and the normal tiles. For example, the threshold rarity metric may be predetermined such that the rare event identification system may identify in a range of 1 to 10 rare tiles, or a range of 1 to 100 rare tiles, or a range of 1 to 1,000 rare tiles, which have rarity index values larger than those of remaining rare tiles and the normal tiles.

In this disclosure, the rare event identification system may further be configured to determine the presence of a rare event in each tile.

In this disclosure, the object may be a human subject. The sample may be a biological sample. The sample may be a liquid and/or solid biological sample.

In this disclosure, the liquid sample may include a body fluid sample of the subject. The liquid sample may include a blood sample, a bone marrow sample, a peritoneal fluid sample, a urine sample, a saliva sample, a vaginal fluid sample, a semen sample, a tear sample, a mucus sample, an aqueous humor sample, cerebrospinal fluid (CSF) sample, or a combination thereof. The liquid sample may include a blood sample.

In this disclosure, the subject may be a cancer patient. The cancer may include breast cancer, bladder cancer, prostate cancer, lung cancer, colon cancer, rectal cancer, kidney cancer, liver cancer, pancreatic cancer, thyroid cancer, leukemia, melanoma, or a combination thereof.

In this disclosure, the rare events may include or may be images of cancer cells that have cancer genomic profiles and/or cancer protein markers; or tumor microenvironment cells that may leak into circulation, wherein these cells may include epithelial cells, endothelial cells, mesenchymal cells, other stromal cells, cells that may be in various transitional states, or a mixture thereof; or immune cells that may be responding to the tumor itself or cancer treatment; or extra-cellular vesicles; or a mixture thereof. The rare events may include or may be images of conventional circulating tumor cells, which may be CK+, vimentin-, CD31− and CD45−; or circulating tumor cells, which may be CK+, CD31−, CD45−, and vimentin+, and wherein tumor cells may putatively in epithelial to mesenchymal transition; or tumor cells, which may be CK+, and coated with platelets, which may be CD31+; or endothelial cells, which may be CD31+, vimentin+, and CK−; or endothelial cells, which may be CD31+, vimentin+ and CK+; or megakaryocytes, which may be CD31+ and vimentin−, wherein megakaryocytes may include large cells containing a single, large, multi− lobulated, polyploidy nucleus responsible for the production of blood thrombocytes platelets; or large cells, which may be CD31+, and cytokeratins that are CK+, wherein these large cells may be present in the liquid biopsy samples obtained from a bone marrow; or cells, which may be DAPI+ and vimentin+; or round cells, which may be CD45+ and CK+; round cells, which may be CD45+, vimentin+, CD45+, and CK+; clusters of cells including at least two cells, wherein the cells may be of same type of cells and/or different types of cells; or cells, which are DAPI+, CD45−, CD31−, and CK−; or immune cells, which may be CD45+ and vimentin−; or immune cells, which may be CD45+ and vimentin+ (type III intermediate filament protein); extra− cellular vesicles; or a mixture thereof.

In this disclosure, the rare event identification system may include an optical imaging system and a processing system. The rare event identification system may further be configured to generate the sample image. The sample image may be generated using a fluorescence imaging system. The fluorescence imaging system may have at least one fluorescence channel. Each fluorescence channel may be configured to generate at least one sample image.

In this disclosure, the rare event identification system may include an optical imaging system and a processing system. The optical imaging system may be a fluorescence imaging system. The optical imaging system may include at least one fluorescence channel.

In this disclosure, the number of fluorescence channels may be in a range of 1 to 10, or a range of 4 to 7. Such optical imaging systems may further be configured to generate the sample image. The fluorescence imaging system may be configured to identify a biological structure.

In this disclosure, the biological structure is a biological structure with a membrane, a protein, DNA, RNA, or a combination thereof; wherein the biological structure with a membrane is a cell, an extra-cellular vesicle, or a combination thereof. The extra-cellular vesicle may be an oncosome. The oncosome may have a characteristic size larger than that of an exosome. The oncosome may have a characteristic size equal to or larger than one micrometer. Further examples of the biological structure may include a cell nucleus, an epithelial cell, a white blood cell, an endothelial cell, a mesenchymal cell, an extra-cellular vesicle, or a combination thereof.

In this disclosure, the liquid sample may be stained with an immunofluorescent antibody formulation (i.e., an assay). The immunofluorescent antibody formulation may, for example, include a fluorescence stain to label a cell nucleus, an immunofluorescent antibody to label an epithelial cell, an immunofluorescent antibody to label a white blood cell, an immunofluorescent antibody to label an endothelial cell, an immunofluorescent antibody to label a mesenchymal cell, or a combination thereof.

In this disclosure, the optical imaging system may be configured to illuminate the liquid sample and detect emitted electromagnetic radiation from the liquid sample. The optical imaging system may include a liquid biopsy sample carrier suitable for receiving and supporting the liquid sample; an illumination system capable of illuminating the liquid sample at a specific wavelength or wavelengths that can be absorbed by at least one fluorophore (e.g., immunofluorescent dye); a light detection system configured to detect and determine intensity and a wavelength of fluorescence emitted by the at least one fluorophore; and a light controlling system configured to allow detection of emitted electromagnetic radiation from the liquid biopsy sample; allow detection of electromagnetic radiation scattered by, reflected by, and/or transmitted through the liquid biopsy sample; and guide electromagnetic radiation from the illumination system to the liquid sample, and from the liquid sample to the light detection system. The emitted electromagnetic radiation may be fluorescent radiation. The optical imaging system may include an excitation filter, an emission filter, a (dichroic) mirror, a lens, an optical fiber, or a combination thereof. The optical imaging system may include a fluorescence microscope, a brightfield microscope, or a combination thereof.

In this disclosure, the rare event identification system is configured to obtain a plurality of sample images or process data related to the plurality of tiles sectioned from a plurality of sample images. These sample images may be formed by using a fluorescence spectroscopy system and/or a bright field imaging system (e.g., a bright field microscope). The rare event identification system(s) of this disclosure is configured to generate, use, obtain, and/or process data related to one or more sample images formed by using a one or multi-channel fluorescence spectroscopy system and/or a bright field imaging system. In one example of such rare event identification system(s) of this disclosure, the rare event identification system is configured to obtain a plurality of sample images, wherein: (a) each sample image of a group of sample images may be formed by using a different fluorescence channel dedicated to identifying a specific marker(s) associated with a biological structure; and (b) the sample image group comprises at least 2 sample images, or at least 3 sample images, or at least 4 sample images, or at least 5 sample images, or at least 6 sample images, or at least 7 sample images, or at least 8 sample images, or at least 9 sample images, or at least 10 sample images. In another example such rare event identification system(s) of this disclosure, the rare event identification system is configured to obtain a plurality of sample images, wherein: (a) at least one sample image is formed by using a bright-field imaging system; (b) each sample image of a group of sample images is formed by using a different fluorescence channel dedicated to identifying a specific marker(s) associated with a biological structure; and (c) the sample image group comprises at least 2 sample images, or at least 3 sample images, or at least 4 sample images, or at least 5 sample images, or at least 6 sample images, or at least 7 sample images, or at least 8 sample images, or at least 9 sample images, or at least 10 sample images.

In this disclosure, the rare event identification system may further be configured to determine the presence of a rare event in each tile.

In this disclosure, the processing system may include a control system, a hardware processor, a memory system, and an information-conveying system.

In this disclosure, the rare event identification system performs end-to-end adaptive imaging and learning, in which the optical imaging system and processing system function cooperatively to acquire sample images, section the images into tiles, train an autoencoder on the acquired data, compute reproducibility differences, and adjust illumination or spectral or magnification parameters of the imaging hardware in response to identified rare or information-rich regions. This embodiment provides a fully integrated learning-and-control platform.

In this disclosure, the rare event identification system operates as a model-training apparatus that uses tile data obtained from sample images to train the autoencoder until it learns to reproduce each tile, determine reproducibility differences, and rank the tiles. In this configuration, the system may optionally provide feedback to imaging parameters during the training process but does not require an already trained model. This embodiment thus establishes the machine-learning framework and generates a trained autoencoder model for later use.

In this disclosure, the rare event identification system functions as a model-inference or deployment apparatus that receives sample images and applies a pre-trained autoencoder to identify, quantify, and rank rare tiles based on reproducibility differences. The system may further command the optical imaging components to focus, re-image, or adjust illumination or magnification parameters in regions identified as rare or biologically relevant. This embodiment therefore provides a hardware-implemented inference system for automated detection and visualization of rare events using an existing trained model.

The scope of this disclosure includes any combination of inventive features disclosed in the preceding paragraphs of this section or the paragraphs of the following sections.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings are illustrative examples. They do not illustrate all examples. Other examples may be used in addition or instead. Details that may be apparent or unnecessary may be omitted to save space or for more effective illustration. Some examples may be practiced with additional components or steps and/or without all of the components or steps that are illustrated. When the same numeral appears in different drawings, it refers to the same or like components or steps.

For a further understanding of the nature, objects, and advantages of the present disclosure, reference should be made to the following detailed description, read in conjunction with the following drawings, wherein like reference numerals denote like elements and wherein the following drawings are not necessarily to scale.

FIG. 1. A block illustration of an exemplary biological structure identification system.

FIG. 2. A schematic illustration of an exemplary biological structure identification system.

FIG. 3. A block diagram of an exemplary approach for rare event detection. In this illustration, the IF image is an immunofluorescence (IF) image, and the WAE is a Wasserstein Autoencoder (WAE).

FIG. 4. A block diagram of an exemplary Wasserstein Autoencoder (WAE) architecture. Note that the numerical labels under the blocks represent the dimensions of the output of each block.

FIG. 5. A block diagram of an exemplary encoder architecture. Note that the numerical labels under the blocks represent the dimensions of the output of each block. Layer normalization is used within the dense blocks, as shown in FIG. 6.

FIG. 6. A block diagram of an exemplary decoder architecture. Note that the numerical labels under the blocks represent the dimensions of the output of each block. Batch normalization is used within the dense blocks, as shown in FIG. 7.

FIG. 7. A block diagram of an exemplary dense architecture. Note that the numerical labels under the blocks represent the dimensions of the output of each block.

FIG. 8. A block diagram of an exemplary convolution architecture. Note that the numerical labels under the blocks represent the dimensions of the output of each block.

FIG. 9. A block diagram of an exemplary up-sample architecture. Note that the numerical labels under the blocks represent the dimensions of the output of each block.

FIG. 10. A block diagram of an exemplary down-sample block architecture. Note that the numerical labels under the blocks represent the dimensions of the output of each block.

FIG. 11. Six “normal-tile” images and six “rare-tile” images belong to Dataset-1, with 150 normal-tile images and 30 rare-tile images.

FIG. 12. Six normal-tile images and six rare-tile images belong to Dataset-2, with 150 normal-tile images and 30 rare-tile images.

FIG. 13. illustrates an exemplary normal-tile result for Dataset-1: The first column of three images is the normal-tile's composite color image of the four channels, and the subsequent three columns of three images are the normal-tile's grayscale intensity images in each of the four channels. The top row is the true images obtained by immunofluorescence imaging, the middle row of five images were images reconstructed by running a WAE, and the bottom row of five images were images obtained by subtracting the images reconstructed by running a WAE from the true images obtained by immunofluorescence imaging, which is the reproducibility difference.

FIG. 14. illustrates an exemplary rare-tile result for Dataset-1: The first column of three images is the rare-tile's composite color image of the four channels, and the subsequent three columns of three images are the rare-tile's grayscale intensity images in each of the four channels. The top row is the true images obtained by immunofluorescence imaging, the middle row of five images were images reconstructed by running a WAE, and the bottom row of five images were images obtained by subtracting the images reconstructed by running a WAE from the true images obtained by immunofluorescence imaging, which is the reproducibility difference.

FIG. 15. illustrates an exemplary normal-tile result for Dataset-2: The first column of three images is the normal-tile's composite color image of the four channels, and the subsequent three columns of three images are the normal-tile's grayscale intensity images in each of the four channels. The top row is the true images obtained by immunofluorescence imaging, the middle row of five images were images reconstructed by running a WAE, and the bottom row of five images were images obtained by subtracting the images reconstructed by running a WAE from the true images obtained by immunofluorescence imaging, which is the reproducibility difference.

FIG. 16. illustrates an exemplary rare-tile result for Dataset-2: The first column of three images is the rare-tile's composite color image of the four channels, and the subsequent three columns of three images are the rare-tile's grayscale intensity images in each of the four channels. The top row is the true images obtained by immunofluorescence imaging, the middle row of five images were images reconstructed by running a WAE, and the bottom row of five images were images obtained by subtracting the images reconstructed by running a WAE from the true images obtained by immunofluorescence imaging, which is the reproducibility difference.

FIG. 17A-B. ROC curves of the classification resulting from the WAE ranking for Dataset 1 (left) and Dataset 2 (right).

FIG. 18. Schematically illustrates an exemplary rare event identification system of this disclosure.

FIG. 19. Schematically illustrates an exemplary rare event identification system of this disclosure.

FIG. 20. Schematically illustrates an exemplary rare event identification system of this disclosure.

FIG. 21. Schematically illustrates an exemplary rare event identification system of this disclosure.

FIG. 22. Schematically illustrates an exemplary rare event identification system of this disclosure.

FIG. 23. Schematically illustrates an exemplary rare event identification system of this disclosure.

### DETAILED DESCRIPTION

Reference will now be made in detail to presently preferred compositions, embodiments, and methods of the present invention, constituting the best modes of practicing the invention currently known to the inventors. However, it is to be understood that the disclosed embodiments are merely exemplary of the invention that may be embodied in various and alternative forms. Therefore, specific details disclosed herein are not to be interpreted as limiting but merely as a representative basis for any aspect of the invention and/or as a representative basis for teaching one skilled in the art to employ the present invention in various ways.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary, percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It must also be noted that, as used in the specification and the appended claims, the singular form “a,” “an,” and “the” comprise plural referents unless the context clearly indicates otherwise. For example, reference to a component in the singular is intended to include a plurality of components.

As used herein, the term “about” means that the amount or value in question may be the specific value designated or some other value in its neighborhood. Generally, the term “about” denoting a specific value is intended to denote a range within +/−5% of the value. As one example, the phrase “about 100” denotes a range of 100+/−5, i.e., the range from 95 to 105. Generally, when the term “about” is used, it can be expected that similar results or effects according to the invention can be obtained within a range of +/−5% of the indicated value.

As used herein, the term “and/or” means that either all or only one of the elements of said group may be present. For example, “A and/or B” shall mean “only A, or only B, or both A and B.” In the case of “only A,” the term also covers the possibility that B is absent, i.e., “only A, but not B.”

It is also to be understood that this invention is not limited to the specific embodiments and methods described below, as specific components and/or conditions may vary. Furthermore, the terminology used herein is used only to describe particular embodiments of the present invention and is not intended to be limiting in any way.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element outlined in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The phrase “composed of” means “including” or “comprising Typically, this phrase denotes that an object is formed from a material.

Concerning the terms “comprising,” “consisting of,” and “consisting essentially of,” where one of these three terms is used herein, the presently disclosed and claimed subject matter can include the use of either of the other two terms.

The term “one or more” means “at least one,” and the term “at least one” means “one or more.” The terms “one or more” and “at least one” include “plurality” as a subset.

In this disclosure, the indefinite article “a” and the phrases “one or more” and “at least one” are synonymous and mean “at least one.”

Relational terms such as “first” and “second” and the like may be used solely to distinguish one entity or action from another without necessarily requiring or implying any actual relationship or order between them. The terms “comprises,” “comprising,” and any other variation when used in connection with a list of elements in the specification or claims are intended to indicate that the list is not exclusive and that other elements may be included. Similarly, an element preceded by an “a” or an “an” does not, without further constraints, preclude the existence of additional elements of the identical type.

The terms “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within +0%, 0.1%, 0.5%, 1%, 2%, 3%, 4%, 5% or 10% of the value or relative characteristic.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 10 . . . 20 . . . 50 . . . 76 . . . 83 . . . 97, 98, 99, 100. Similarly, when any range is called for, intervening numbers that are increments of the difference between the upper and lower limits divided by 10 can be taken as alternative upper or lower limits. For example, if the range is 1.1. to 2.1, the following numbers: 1.2, 1.3, 1.4, 1.5, 1.6, 1.7, 1.8, 1.9, and 2.0 can be selected as lower or upper limits.

The disclosure “International Application (PCT) No. PCT/US23/11889, Liquid Biopsy Analytes to Define Cancer Stages,” filed Jan. 30, 2023, is hereby incorporated in its entirety by reference herein. The publication by Setayesh et al. “Multianalyte liquid biopsy to aid the diagnostic workup of breast cancer. NPJ Breast Cancer. 2022 Sep. 27; 8 (1): 112. doi: 10.1038/s41523-022-00480-4. PMID: 36167819; PMCID: PMC9515081” and the supplemental information of this publication is herein incorporated in its entirety by reference.

In the examples set forth herein, concentrations, temperature, measurement conditions, and reaction conditions (e.g., pressure, pH, and temperature) can be practiced with plus or minus 50 percent of the values indicated rounded to or truncated to two significant figures of the value provided in the examples. In a refinement, concentrations, temperature, and reaction conditions (e.g., pressure, pH, and temperature) can be practiced with plus or minus 30 percent of the values indicated rounded to or truncated to two significant figures of the value provided in the examples. In another refinement, concentrations, temperature, and reaction conditions (e.g., pressure, pH, temperature) can be practiced with plus or minus 10 percent of the values indicated rounded to or truncated to two significant figures of the value provided in the examples.

The term “computing device” generally refers to any device that can perform at least one function, including communicating with another computing device.

When a computer or other computing device is described as performing an action or method step, it is understood that the computer or other computing device is operable to and/or configured to perform the action or method step, typically by executing one or more lines of source code. The actions or method steps can be encoded onto non-transitory memory (e.g., hard drives, optical drives, flash drives, and the like).

The term “configured to or operable to” means that the processing circuitry (e.g., a computer or computing device) is configured or adapted to perform one or more of the actions set forth herein by software configuration and/or hardware configuration. The terms “configured to” and “operable to” can be used interchangeably.

The processes, methods, or algorithms disclosed herein can be delivered to/implemented by a processing device, controller, or computer, including any existing programmable electronic control unit or dedicated electronic control unit. Similarly, the processes, methods, or algorithms can be stored as data and instructions executable by a controller or computer in many forms, including, but not limited to, information permanently stored on non-writable storage media such as ROM devices and information alterably stored on writeable storage media such as floppy disks, magnetic tapes, CDs, RAM devices, and other magnetic and optical media. The processes, methods, or algorithms can also be implemented in an executable software object. Alternatively, the processes, methods, or algorithms can be embodied in whole or in part using suitable hardware components, such as Application Specific Integrated Circuits (ASICs), Field-Programmable Gate Arrays (FPGAs), state machines, controllers, or other hardware components or devices, or a combination of hardware, software, and firmware components.

In this disclosure, where publications, patents, or published patent applications are referenced, the disclosures of these publications, patents, or published patent applications in their entirety are hereby incorporated by reference into this application to more fully describe the state of the art to which this invention pertains.

### ABBREVIATIONS

“Ab” means antibody.
“ASICs” means application-specific integrated circuits.
“AUC” means area under curve.
“BC” means breast cancer.
“BCMA” means B-cell maturation antigen.
“BCT” means blood collection tube (as in Cell-free DNA BCT).
“BMI” means body mass index.
“BSA” means bovine serum albumin.
“BUN” means blood urea nitrogen.
“CD138” means cluster of differentiation 138.
“CD31” means marker for endothelial cells.
“CD45” means marker for immune cells.
“CK” means cytokeratin.
“CK19” means cytokeratin 19.
“CNN” means convolutional neural network.
“CS” means clinical staging.
“CSF” means cerebrospinal fluid.
“CSI-Cancer” means Convergent Science Institute in Cancer.
“DAPI” means 4′,6-diamidino-2-phenylindole.
“EMT” means epithelial to mesenchymal transition.
“ER” means estrogen receptor.
“EV” means extravesical.
“Fab” means fragment antigen binding.
“HCT” means hematocrit.
“Hgb” means hemoglobin.
“IF” means immunofluorescence.
“IgG” means immunoglobulin G.
“mAb” means monoclonal antibody.
“MMD” means maximum mean discrepancy.
“N+” means node positive.
“OC” means organ confined.
“OPTICOLL” means OPTImization of blood COLLection.
“PanCK” means pan-cytokeratin.
“PR” means progesterone receptor.
“PS” means pathological staging.
“PSOC” means Physical Sciences in Oncology.
“ReLU” means rectified linear unit.
“RNA” means ribonucleic acid.
“ROC” means receiver operating characteristic.
“ROM” means read-only memory.
“VAE” means variational auto-encoder.
“WAE” means Wasserstein autoencoder.
“WBC” means white blood cell.

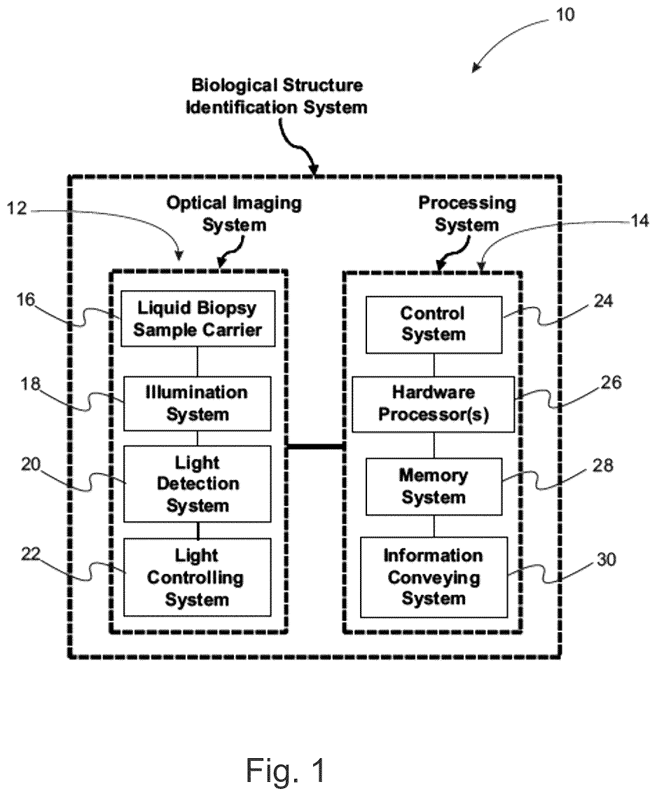

This disclosure relates to a liquid biopsy system, which is hereafter referred to as a “rare event identification system.” An exemplary rare event identification system is shown in FIG. 1. This disclosure also relates to a rare event identification system and a method that can be used to determine a subject's disease state. This disease may be any disease. This disease may be cancer. This disclosure also relates to a method that uses the rare event identification system to propose a treatment strategy for the subject.

In one example, the rare event identification system may be configured to obtain an image of a liquid sample belonging to the subject.

In this disclosure, the image of the sample (“sample image’) may include at least one event. The event may be a common event, a rare event, or a biologically non-relevant event. The common event may be an image of a biological structure ordinarily expected to be observed on an image of a liquid sample belonging to a subject who does not have cancer. The rare event may be an image of a rare biological structure ordinarily expected not to be observed on an image of a liquid sample belonging to a subject who does not have cancer. The biologically non-relevant event may be an image of a non-biological structure and/or an image of a biological event that is not expected to be present in the sample image. The non-biological structure may be any non-biological structure. For example, the non-biological structure may be an artifact such as a dust speck or an immunofluorescent dye aggregate unrelated to a biological structure. For example, the biological event that is not expected to be present in the sample image may be an event incorporated during the preparation of the sample for imaging, such as a biological structure originating from a human who prepares the sample for imaging. The common biological structure may not be indicative of cancer. The rare biological structure may be potentially indicative of cancer.

In one example, the rare event identification system may be configured to generate/obtain an image of a (e.g., liquid) sample; section each sample image into a plurality of tiles; initiate an (e.g., Wasserstein) autoencoder to process data related to tiles; use data related to a number or all tiles to train the autoencoder such that the autoencoder learns to reproduce each tile; determine a reproducibility difference for each tile; and rank each tile according to the reproducibility difference of each tile.

In one example, the rare event identification system may be configured to initiate an autoencoder to process data related to a plurality of tiles; use data related to a number or all tiles to train the autoencoder such that the autoencoder learns to reproduce each tile; determine a reproducibility difference for each tile; and rank each tile according to the reproducibility difference of each tile.

In one example, the rare event identification system may be configured to obtain an image of a sample; section each sample image into a plurality of tiles; provide a trained autoencoder; initiate the trained autoencoder to process data related to the tiles; determine a reproducibility difference for each tile; and rank each tile according to the reproducibility difference of each tile.

In one example, the rare event identification system may be configured to provide a trained autoencoder; initiate the trained autoencoder to process data related to a plurality of tiles; determine a reproducibility difference for each tile; and rank each tile according to the reproducibility difference of each tile.

In this disclosure, the rare event identification system may include an optical imaging system and a processing system. The rare event identification system may further be configured to generate the image of a liquid sample belonging to the subject. The image of a liquid sample belonging to the subject may be generated by using an (e.g., fluorescence or immunofluorescence) imaging system. The optical imaging system may include a (immuno) fluorescence imaging system. The optical imaging system may include a fluorescence spectroscopy system.

In this disclosure, the optical imaging system may be configured to illuminate the liquid sample and detect emitted electromagnetic radiation from the liquid sample. The optical imaging system may include a liquid biopsy sample carrier suitable for receiving and supporting the liquid sample; an illumination system capable of illuminating the liquid sample at a specific wavelength or wavelengths that can be absorbed by at least one fluorophore (e.g., immunofluorescent dye); a light detection system configured to detect and determine intensity and a wavelength of fluorescence emitted by the at least one fluorophore; and a light controlling system configured to allow detection of emitted electromagnetic radiation from the liquid biopsy sample; allow detection of electromagnetic radiation scattered by, reflected by, and/or transmitted through the liquid biopsy sample; and guide electromagnetic radiation from the illumination system to the liquid sample, and from the liquid sample to the light detection system. The emitted electromagnetic radiation may be fluorescent radiation. The optical imaging system may include an excitation filter, an emission filter, a (dichroic) mirror, a lens, an optical fiber, or a combination thereof. The optical imaging system may include a fluorescence microscope, a brightfield microscope, or a combination thereof.

The optical imaging system may include at least one fluorescence channel, or at least four fluorescence channels, or in a range of 1 fluorescence channel to 10 fluorescence channels, or in a range of 4 fluorescence channels to 7 fluorescence channels, or four fluorescence channels. Each fluorescence channel may be configured to generate at least one image of a liquid sample belonging to the subject. Each fluorescence channel may provide different intensity (e.g., of a pixel) related to each event.

In this disclosure, the (fluorescence) imaging system or at least one channel of this imaging system may be configured to identify a cell nucleus, an epithelial cell, a white blood cell, an endothelial cell, a mesenchymal cell, an extra-cellular vesicle, or a combination thereof. At least one channel may be configured to identify both the white blood cell and the endothelial cell.

In this disclosure, the liquid sample may be stained with an immunofluorescent antibody formulation. The immunofluorescent antibody formulation may include a fluorescent stain to label a cell nucleus, an immunofluorescent antibody to label an epithelial cell, an immunofluorescent antibody to label a white blood cell, an immunofluorescent antibody to label an endothelial cell, an immunofluorescent antibody to label a mesenchymal cell, or a combination thereof.

In this disclosure, the biological structure may be a biological structure with a membrane, a protein, DNA, RNA, or a combination thereof. The biological structure with a membrane may be a cell, an extra-cellular vesicle, or a combination thereof. The extra-cellular vesicle may be an oncosome. The oncosome may have a characteristic size larger than that of an exosome. The oncosome may have a characteristic size equal to or larger than one micrometer.

In this disclosure, the liquid sample may include a body fluid sample of the subject. The liquid sample may include a blood sample, a bone marrow sample, a peritoneal fluid sample, a urine sample, a saliva sample, a vaginal fluid sample, a semen sample, a tear sample, a mucus sample, an aqueous humor sample, cerebrospinal fluid (CSF) sample, or a combination thereof. The liquid sample may include a blood sample. The liquid sample may include non-liquid material.

In this disclosure, the rare events may include or may be images of cancer cells that have cancer genomic profiles and/or cancer protein markers; or tumor microenvironment cells that may leak into circulation, wherein these cells may include epithelial cells, endothelial cells, mesenchymal cells, other stromal cells, cells that may be in various transitional states, or a mixture thereof; or immune cells that may be responding to the tumor itself or cancer treatment; or extra-cellular vesicles; or a mixture thereof. The rare events may include or may be images of conventional circulating tumor cells, which may be CK+, vimentin−, CD31− and CD45−; or circulating tumor cells, which may be CK+, CD31−, CD45−, and vimentin+, and wherein tumor cells may putatively in epithelial to mesenchymal transition; or tumor cells, which may be CK+, and coated with platelets, which may be CD31+; or endothelial cells, which may be CD31+, vimentin+, and CK−; or endothelial cells, which may be CD31+, vimentin+ and CK+; or megakaryocytes, which may be CD31+ and vimentin-, wherein megakaryocytes may include large cells containing a single, large, multi-lobulated, polyploidy nucleus responsible for the production of blood thrombocytes platelets; or large cells, which may be CD31+, and cytokeratins that are CK+, wherein these large cells may be present in the liquid biopsy samples obtained from a bone marrow; or cells, which may be DAPI+ and vimentin+; or round cells, which may be CD45+ and CK+; round cells, which may be CD45+, vimentin+, CD45+, and CK+; clusters of cells including at least two cells, wherein the cells may be of same type of cells and/or different types of cells; or cells, which are DAPI+, CD45−, CD31−, and CK−; or immune cells, which may be CD45+ and vimentin-; or immune cells, which may be CD45+ and vimentin+ (type III intermediate filament protein); extra-cellular vesicles; or a mixture thereof.

In this disclosure, the subject may be a human who has cancer. The cancer may include breast cancer, bladder cancer, prostate cancer, lung cancer, colon cancer, rectal cancer, kidney cancer, liver cancer, pancreatic cancer, thyroid cancer, leukemia, melanoma, or a combination thereof.

In this disclosure, the processing system may include a control system, a hardware processor, a memory system, and an information-conveying system.

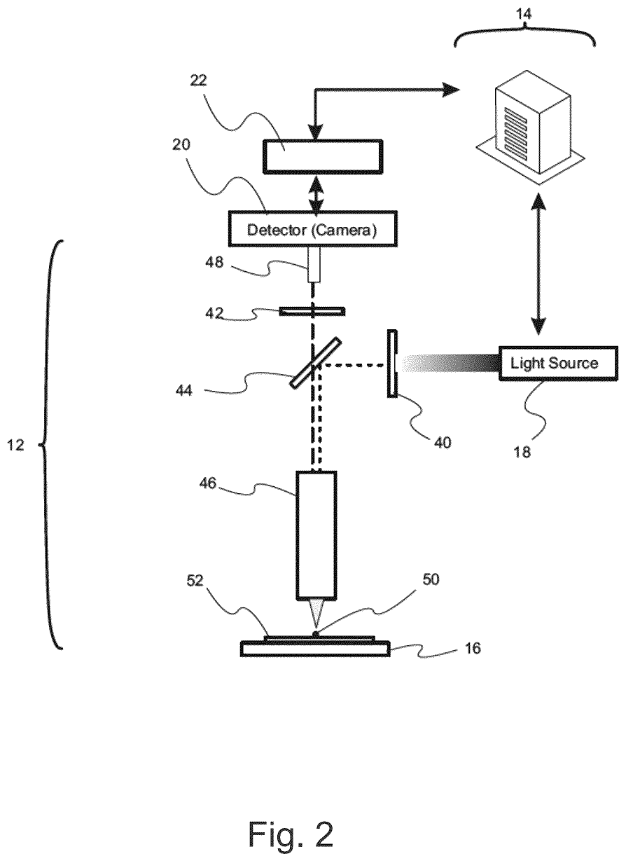

FIG. 1 and FIG. 2 illustrate an exemplary liquid biopsy system for determining a subject's disease (e.g., cancer) state. Such systems may be used to identify biological and non-biological structures in liquids. An image of such structures may form an event(s).

Such biological structure identification system 10 includes an optical imaging system 12 and a processing system 14. The liquid biopsy sample typically includes one or more biological structures that may be labeled with one or more fluorophores. Characteristically, optical imaging system 12 is configured to illuminate a liquid biopsy sample with one or more biological structures labeled with one or more fluorophores associated with a fluorescence assay for cancer, allowing the detection of emitted electromagnetic radiation from the liquid biopsy sample as image data.

In one example, the processing system 14 is configured to (1) generate images of one or more biological structures for the subject from the image data, detect and determine a plurality of features from the images or the image data, and form biological structure identification buckets from the plurality of features, each biological structure identification bucket identifying biological structures that are similar in type; (2) generate a subject profile of biological structure identification buckets for rare biological structures for the subject; (3) compare the subject profile with a set of predetermined cancer stage profiles of subjects having cancer at a plurality of cancer stages; and (4) identify a cancer stage for the subject by determining a predetermined cancer stage profile from the set of predetermined cancer stage profiles to which the subject profile is most similar.

Typically, processing system 14 includes a computing device.

In a variation of the systems of this disclosure, the rare events may be observed as rare biological structures. In another variation, one or more biological structures may include simultaneously identified multiple biological structures. Yet, in another variation, the rare biological structures may be observed as rare imaging events in the imaging data or the image.

As shown in FIG. 1 and FIG. 2, the optical imaging system 12 may include a liquid biopsy sample carrier 16 suitable for supporting the liquid biopsy sample for the identification of the biological structure(s); an illumination system 18 capable of illuminating the liquid biopsy sample at a specific wavelength or wavelengths that the fluorophore can absorb; a light detection system 20 configured to detect and determine intensity and a wavelength of fluorescence emitted by the fluorophore; and a light controlling system 22. The light controlling system 22 can be configured to allow detection of emitted electromagnetic radiation from the liquid biopsy sample; allow detection of electromagnetic radiation scattered by, reflected by, and/or transmitted through the liquid biopsy sample; and guide electromagnetic radiation from the illumination system to the liquid biopsy sample, and from the liquid biopsy sample to the light detection system. Also, as shown in FIG. 2, optical system 22 may include an optical component such as an excitation filter 40, an emission filter 42, a (dichroic) mirror 44, a lens 46, an optical fiber 48, and a combination thereof. FIG. 2 also shows a specimen 50 positioned on a glass slide 52. In a refinement, light from an illumination system 18 (e.g., a laser light source) is passed through an excitation filter 40 and then to a dichroic mirror 44 that directs the excitation light through a lens 46 (e.g., an objective lens). Lens 46 focuses the light onto specimen 50. The resulting emitted or scattered light passes through lens 46, dichroic mirror 44, and emission filter 42. The fluorescent light is then detected by light detection system 20, optionally through fiberoptic 48.

As shown in FIG. 1, the processing system 14 may include a control system 24, a hardware processor 26 (e.g., CPU), a memory system 28, and an information conveying system 30. Processing system 14 will execute the analysis step via hardware processor 26. Control system 24 executes software components that a user uses to control and interact with the optical imaging system 12 and initiate analysis and image construction from the image data received from the optical imaging system. The information conveying system 30 is configured to communicate information to a user, for example, comprising information related to types of biological structures present in the liquid biopsy sample, the disease maps, the disease atlases, or a combination thereof. Control system 24 and information conveying system 30 function via program codes executing on hardware processor 26 and via software and data stored in memory system 28.

In a variation, the rare event identification system 10 is configured to receive a liquid biopsy sample by using the liquid biopsy sample carrier 16 and illuminate the liquid biopsy sample with electromagnetic radiation from illumination system 18 that has a specific wavelength or wavelengths that the fluorophore can absorb. The light detection system 20 is configured to detect and determine the intensity and wavelength of fluorescence emitted by the fluorophore with light detection system 20 or produce input data for these characteristics so that they can be determined by processing system 12.

The processing system 14 is configured to generate an image of the biological structure(s) from image data received from the light detection system 20.

In a refinement of the systems of this disclosure, the optical imaging system may include a fluorescence imaging system, a brightfield imaging system, or a combination thereof. The optical imaging system may include a fluorescence microscope, a brightfield microscope, or a combination thereof.

In this disclosure, the rare event identification system may include at least one fluorescence channel. The number of fluorescence channels may be in a range of 1 to 10 fluorescence channels, or in a range of 4 to 7 fluorescence channels.

In one example, these four fluorescence channels may be a first fluorescence channel configured for detection useful for nuclear segmentation and characterization; a second fluorescence channel configured to detect a cytokeratin (CK) for its epithelial-like phenotype; a third fluorescence channel configured to detect vimentin for its endothelial/mesenchymal-like phenotype; and a fourth fluorescence channel configured to detect both a CD31 for its endothelial-like phenotype and a CD45 for its immune cell phenotype. For example, these four fluorescence channels may be a first fluorescence channel configured to detect fluorescence emission at a blue color wavelength region; a second fluorescence channel configured to detect fluorescence emission at a red color wavelength region; a third fluorescence channel configured to detect fluorescence emission at an orange color wavelength region; and a fourth fluorescence channel configured to detect fluorescence emission at a green color wavelength region. For example, these four regions can be defined by an emission filter centered at 455 nm with a bandwidth of 50 nm for blue color wavelengths, an emission filter centered at 525 nm with a bandwidth of 36 nm for green color wavelengths, an emission filter centered at 605 nm with a bandwidth of 52 nm for orange color wavelengths, and an emission filter centered at 705 nm with a bandwidth of 72 nm for red color wavelengths. The first immunofluorescence channel may be configured to detect 4′,6-diamidino-2-phenylindole (DAPI) for nuclear segmentation and characterization.

This disclosure also includes an immunofluorescence assay for analyzing a liquid biopsy sample. This assay, for example, may include antibodies against cytokeratin (CK), vimentin, CD31, and CD45. In a refinement, at least a subset of the antibodies against cytokeratin (CK), vimentin, CD31, and CD45 are labeled with a fluorophore. In the baseline assay, cytokeratin (CK) and vimentin are each independently labeled with a fluorophore, while one or both CD31 and CD45 are labeled with a fluorophore. Examples of fluorophores may include DAPI and Hoechst 33342 and 33258 (as nuclear dyes), Alexa Fluor 488 (for Vimentin), Alexa Fluor 555 (for cytokeratin), Alexa Fluor 647 (for CD31/CD45), and the like. In one example, a four-channel essay, including such fluorophores/dyes, may be used for the identification of markers such as nucleus at about 390 nm (channel 1), BCMA at about 475 nm (channel 2), CD138 at about 555 nm (channel 3), and CD45 at about 635 nm (channel 4). In another example, a six-channel essay, including such fluorophores/dyes, may be used for the identification of markers such as nucleus at about 390 nm (channel 1), BCMA at about 475 nm (channel 2), cytokeratin at about 555 nm (channel 3), ER at about 575 nm (channel 4), PR at about 635 nm (channel 5), and CD45 at about 748 nm (channel 6).

Rare cells may travel through the circulation with short or long half-lives. They may also include stopovers in various tissues along the way.

Representing the disease may mean that these rare cells may be (a) cancer cells as may be evidenced by their cancer genomic profiles and/or cancer protein markers; (b) tumor microenvironment cells that leak into circulation, wherein these cells may comprise epithelial cells, endothelial cells, mesenchymal cells, other stromal cells, cells that are in various transitional states, or a mixture thereof; (c) immune cells that may be responding to the tumor itself or cancer treatment; or (d) a mixture thereof.

The appearances of categories and classification of rare cells may differ across different cancers and states of each cancer. Systems, methods, and assays of this disclosure may identify various cellular subtypes reproducibly for clinical practice while also enabling the discovery of the unknown with an ability to detect a vast majority that has been implicated simultaneously in a unified experiment.

Cell subclasses may be separated by protein and nuclear patterns and cell morphology. Subclasses may be validated by downstream genomic or proteomic analyses, which might or might not be necessary for future clinical applications.

In another variation of the systems of this disclosure, one example relates to an approach to distinguish a substantially larger number of cellular groups using five markers. These markers are fluorescently protein antibodies or molecules labeled to four distinct fluorophores or fluorescent antibodies. The computational method combines morphological differences revealed by distinct fluorescence signatures to distinguish between at least twelve rare cell subtypes, which may be present in the liquid biopsy sample. These rare cells are listed below.

This approach leverages a new sample processing protocol, reducing the five markers into four fluorescence channels and a novel computational method for classifying the different rare cell types via analysis of fluorescent microscopy images. The choice of marker combinations within and across fluorescent channels is necessary for the success of this approach.

In a variation, an integrated training and control system for rare event identification is provided. Referring to FIGS. 1 and 2, a rare event identification and imaging control system 10 includes an optical imaging system 12 which has a liquid biopsy sample carrier 16 suitable for supporting a liquid biopsy sample. System 10 also includes an illumination system 18 capable of illuminating the liquid biopsy sample at a specific wavelength or wavelengths that a fluorophore can absorb. A light detection system 20 is configured to detect and determine intensity and wavelength of fluorescence emitted by the fluorophore. Light controlling system 22 is configured to allow detection of emitted, scattered, reflected, and/or transmitted electromagnetic radiation from the liquid biopsy sample and to guide electromagnetic radiation from the illumination system to the liquid biopsy sample and from the liquid biopsy sample to the light detection system. The light controlling system 22 comprises at least one of an excitation filter 40, an emission filter 42, a dichroic mirror 44, a lens 46, and an optical fiber 48. The system further includes a processing system 14 including a control system 24, a hardware processor 26, a memory system 28, and an information conveying system 30. The hardware processor 26 is configured to execute instructions stored in the memory system 28 to: (i) receive image data from the light detection system 20 and generate a sample image of biological structure(s) present in the liquid biopsy sample; (ii) section the sample image into a plurality of tiles; (iii) initiate an autoencoder to process data related to the tiles and use data related to a number or all of the tiles to train the autoencoder such that the autoencoder learns to reproduce each tile; (iv) for each tile, define original tile's data as input to the autoencoder and reproduced tile's data as output from the autoencoder, determine a reproducibility difference between the original tile's data and the reproduced tile's data, and rank the tiles according to their reproducibility differences; and (v) responsive to identifying, within a region of the sample image comprising multiple tiles, a condition in which a proportion of tiles in the region have reproducibility differences that meet or exceed a predetermined criterion, command, via the control system 24, at least one of: (v-a) the illumination system 18 to reduce illumination intensity and/or to select a reduced subset of illumination wavelengths; (v-b) the light controlling system 22 to select corresponding spectral channels by configuring at least one of the excitation filter 40 and the emission filter 42; and (v-c) to change the magnification of the imaging system. The information conveying system 30 is configured to communicate to a user information including locations of tiles ranked as rare on the sample image.

In a variation, an integrated training system for rare event identification is provided. Referring to FIGS. 1 and 2, a rare event identification and imaging control system 10 includes an optical imaging system 12 including a liquid biopsy sample carrier 16 suitable for supporting a liquid biopsy sample, an illumination system 18 capable of illuminating the liquid biopsy sample at a specific wavelength or wavelengths that a fluorophore can absorb, a light detection system 20 configured to detect and determine intensity and wavelength of fluorescence emitted by the fluorophore, and a light controlling system 22, the light controlling system 22 comprising at least one of an excitation filter 40, an emission filter 42, a dichroic mirror 44, a lens 46, and an optical fiber 48. The system also includes a processing system 14 including a control system 24, a hardware processor 26, a memory system 28, and an information conveying system 30, wherein the hardware processor 26 is configured to execute instructions stored in the memory system 28 to: receive image data from the light detection system 20 and generate a sample image of biological structure(s) present in the liquid biopsy sample; section the sample image into a plurality of tiles, the plurality of tiles being formed by sectioning the sample image into a sufficient number of tiles to train an autoencoder; initiate an autoencoder to process data related to the tiles and use data related to a number or all of the tiles to train the autoencoder such that the autoencoder learns to reproduce each tile; for each tile, define original tile's data as input to the autoencoder and reproduced tile's data as output from the autoencoder, determine a reproducibility difference between the original tile's data and the reproduced tile's data, and rank the tiles according to their reproducibility differences; and responsive to identifying, during training, within a region of the sample image comprising multiple tiles, that a proportion of tiles have reproducibility differences meeting or exceeding a predetermined training rarity criterion, command, via the control system 24, at least one of: the illumination system 18 to select a reduced subset of illumination wavelengths for subsequent acquisition of that region; to change the magnification of the imaging system; and the light controlling system 22 to select corresponding spectral channels by configuring at least one of the excitation filter 40 and the emission filter 42 for subsequent acquisition of that region, wherein the information conveying system 30 is configured to communicate to a user information including locations of tiles ranked as rare on the sample image.

In a variation, an integrated inference system for rare event identification is provided. Referring to FIGS. 1 and 2, a rare event identification and imaging control system 10 includes an optical imaging system 12 including a liquid biopsy sample carrier 16, an illumination system 18 capable of illuminating a liquid biopsy sample at a specific wavelength or wavelengths that a fluorophore can absorb, a light detection system 20 configured to detect and determine intensity and wavelength of fluorescence emitted by the fluorophore, and a light controlling system 22, the light controlling system 22 comprising at least one of an excitation filter 40, an emission filter 42, a dichroic mirror 44, a lens 46, and an optical fiber 48. The system also includes a processing system 14 including a control system 24, a hardware processor 26, a memory system 28, and an information conveying system 30, wherein the hardware processor 26 is configured to execute instructions stored in the memory system 28 to: receive image data from the light detection system 20 and generate a sample image of biological structure(s) present in the liquid biopsy sample; section the sample image into a plurality of tiles; provide a trained autoencoder and initiate the trained autoencoder to process data related to the tiles; for each tile, define original tile's data as input to the trained autoencoder and reproduced tile's data as output from the trained autoencoder, determine a reproducibility difference between the original tile's data and the reproduced tile's data, and rank the tiles according to their reproducibility differences; and responsive to identifying, within a region of the sample image comprising multiple tiles, that a proportion of tiles have reproducibility differences meeting or exceeding a predetermined rarity criterion, command, via the control system 24, at least one of: the illumination system 18 to reduce illumination intensity and/or to select a reduced subset of illumination wavelengths; to change the magnification of the imaging system; and the light controlling system 22 to select corresponding spectral channels by configuring at least one of the excitation filter 40 and the emission filter 42, wherein the information conveying system 30 is configured to communicate to a user information including locations of tiles ranked as rare on the sample image.

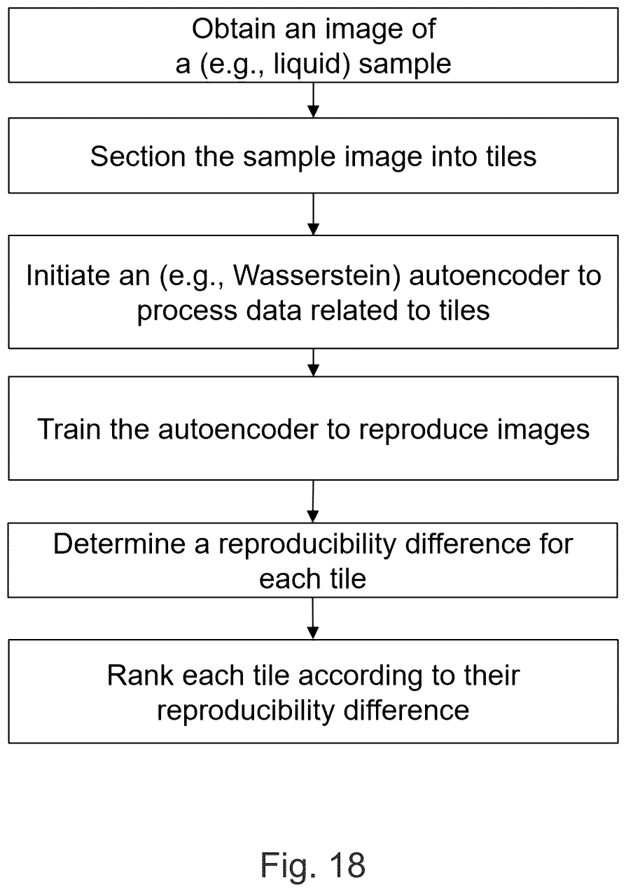

Examples of rare event identification systems are schematically illustrated in FIGS. 18-23.

An exemplary rare event identification system for the determination of a subject's disease state is illustrated in FIG. 18. This exemplary system may be configured to generate/obtain an image of a (e.g., liquid) sample; section each sample image into a plurality of tiles; initiate an (e.g., Wasserstein) autoencoder to process data related to tiles; use data related to a number or all tiles to train the autoencoder such that the autoencoder learns to reproduce each tile; determine a reproducibility difference for each tile; and rank each tile according to the reproducibility difference of each tile. This sample is derived (e.g., obtained) from an object, such as a human subject.

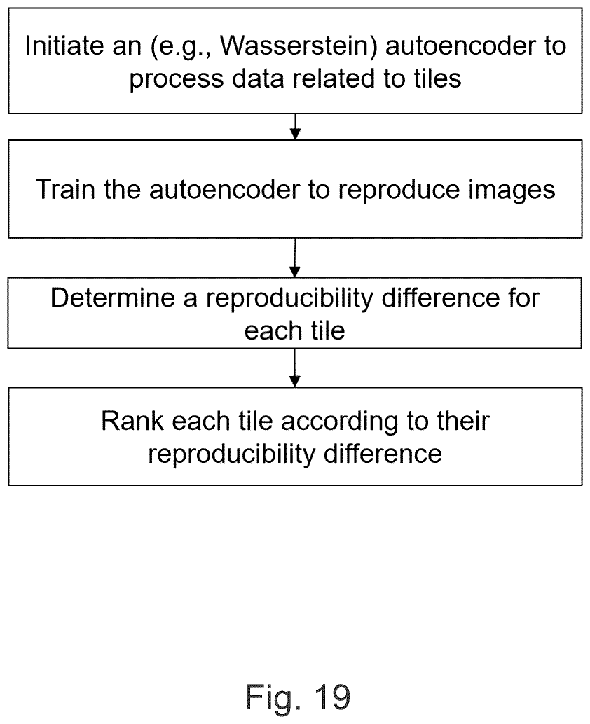

Another exemplary rare event identification system for the determination of a subject's disease state is illustrated in FIG. 19. This exemplary system may be configured to initiate an (e.g., Wasserstein) autoencoder to process data related to tiles; use data related to a number or all tiles to train the autoencoder such that the autoencoder learns to reproduce each tile; determine a reproducibility difference for each tile; and rank each tile according to the reproducibility difference of each tile.

The image of the sample is hereafter referred to as the sample image. The sample image includes a plurality of pixels and the intensity of each of said pixels. The tile is a sectioned image of the sample image. The sample image is sectioned into a sufficient number of tiles to train the autoencoder. The tile's data that is input to the autoencoder is hereafter referred to as the original tile's data. The tile's data output from the autoencoder is hereafter referred to as the reproduced tile's data. The reproducibility difference is the difference between each original tile's data and a reproduced tile's data.

In this disclosure, the rare event identification system may further be configured to identify rare tiles that may not be relevant to the object from which the sample is obtained. The non-relevant tiles may contain events that may be erroneously introduced into the sample or the sample image or the tile image during the obtaining or processing the sample image or the tile image. In this disclosure, said non-relevant tiles are referred to as biologically non-relevant tiles. For example, the biologically non-relevant tiles may contain events that may be introduced as artifacts and/or due to sampling errors and/or sample processing errors and/or system errors.

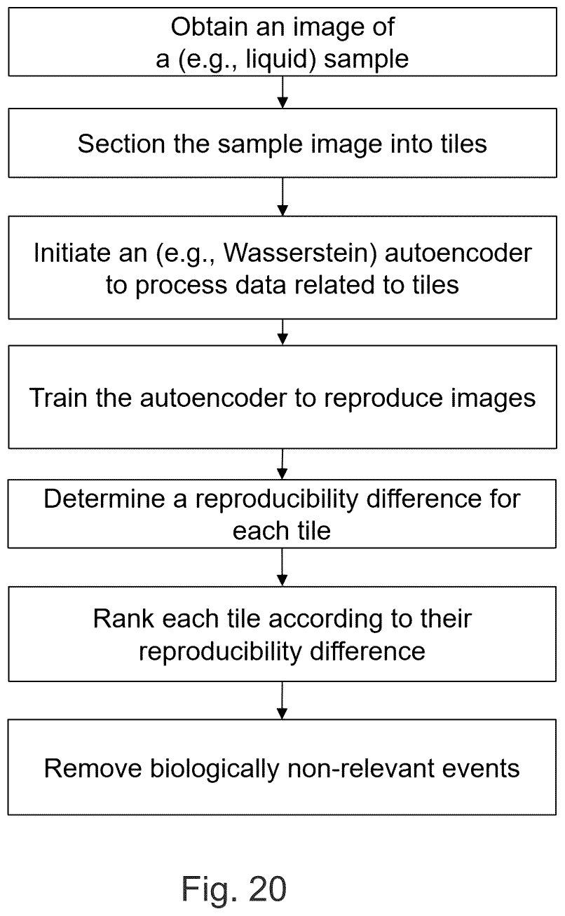

An exemplary rare event identification system for the determination of a disease state of a subject is illustrated in FIG. 20. This exemplary system may be configured to generate/obtain an image of a (e.g., liquid) sample; section each sample image into a plurality of tiles; initiate an (e.g., Wasserstein) autoencoder to process data related to tiles; use data related to a number or all tiles to train the autoencoder such that the autoencoder learns to reproduce each tile; determine a reproducibility difference for each tile; and rank each tile according to the reproducibility difference of each tile. This exemplary system is further configured to remove biologically non-relevant events. This sample may be derived (e.g., obtained) from an object, such as a human subject.

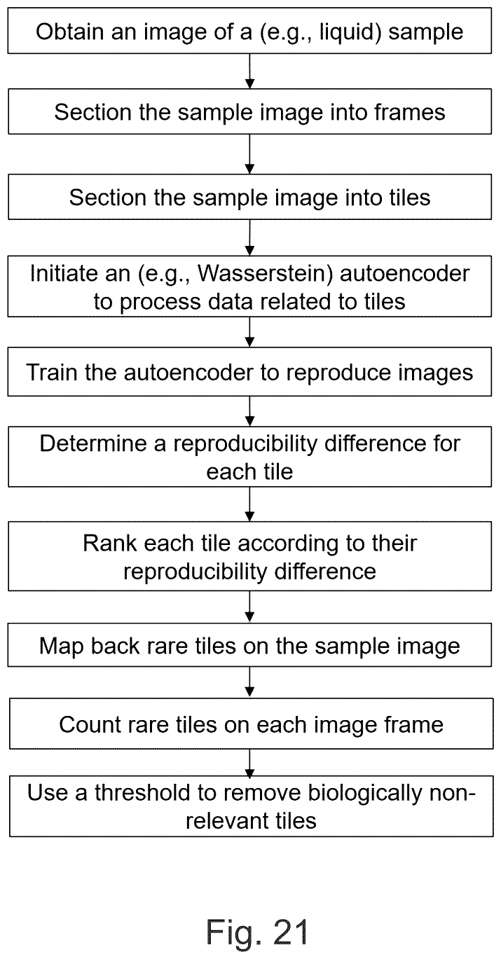

An exemplary rare event identification system for the determination of a subject's disease state is illustrated in FIG. 21. This exemplary system may be configured to generate/obtain an image of a (e.g., liquid) sample; section each image into image frames; section each sample image into a plurality of tiles; initiate an (e.g., Wasserstein) autoencoder to process data related to tiles; use data related to a number or all tiles to train the autoencoder such that the autoencoder learns to reproduce each tile; determine a reproducibility difference for each tile; rank each tile according to the reproducibility difference of each tile; map back each rare tile back to its spatial location on the sample image; count the number of rare tiles on each image frame; and remove rare tiles from said image frame if a number of said rare tiles present in one image frame exceeds a predetermined threshold rare tile number. The threshold rare tile number may be at least ten rare tiles, or 100 rare tiles, or 1000 rare tiles in one image frame said tiles belong. The size of each image frame is larger than that of a tile. For example, the number of tiles in one frame may be in a range of 100 to 1,000, or a range of 100 to 10,000, or a range of 100-100,000, or a range of 500 to 5000. For example, the number of tiles in one frame may be in the range of 500 to 5000. In this example, the sample may be derived (e.g., obtained) from an object, such as a human subject.

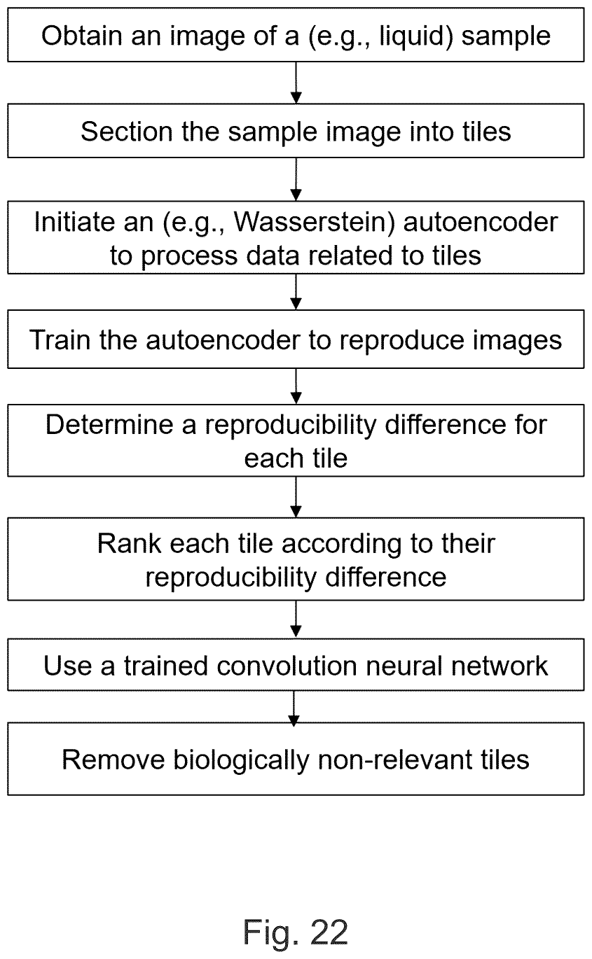

An exemplary rare event identification system for the determination of a disease state of a subject is illustrated in FIG. 22. This exemplary system may be configured to generate/obtain an image of a (e.g., liquid) sample; section each sample image into a plurality of tiles; initiate an (e.g., Wasserstein) autoencoder to process data related to tiles; use data related to a number or all tiles to train the autoencoder such that the autoencoder learns to reproduce each tile; determine a reproducibility difference for each tile; rank each tile according to the reproducibility difference of each tile; use a trained convolutional neural network (CNN); and remove biologically non-relevant tiles. The trained convolutional neural network may be obtained by training a CNN on two sets of tiles. The first training tile set may include images of biologically non-relevant events; and the second training tile set may include only images of relevant events. In this example, the sample may be derived (e.g., obtained) from an object, such as a human subject.

In one example, the trained CNN may be obtained by training a CNN on two sets of training tiles. The first training tile set may include images of biologically non-relevant events, and the second training tile set may include only images of relevant events. The CNN may be trained by obtaining a sample image sectioned into a plurality of tiles, each tile containing data representing a portion of the sample image; creating a first set of training tiles comprising images of biologically non-relevant events; creating a second set of training tiles comprising images of biologically relevant events; training the CNN on the first and second sets of training tiles to classify each tile as either biologically relevant or biologically non-relevant based on its learned features; and thereby obtaining a trained CNN. The CNN may be trained to classify tiles based on features extracted from intensity values across multiple channels of the sample image, wherein each channel is associated with a specific biomarker. The rare event identification system may be further configured to apply the trained CNN to a new set of tiles obtained from a sample image and to remove tiles classified as biologically non-relevant, thereby isolating tiles with biologically relevant events. The rare event identification system may be further configured to train a CNN on a first set of training tiles and a second set of training tiles to obtain a trained CNN, wherein the first set of training tiles comprises tiles with biologically non-relevant events; and wherein the second set of training tiles comprises tiles with biologically relevant events; and apply the trained CNN to remove tiles classified as biologically non-relevant, thereby isolating tiles with biologically relevant events for further analysis. The CNN may be trained with a supervised learning approach using a loss function that minimizes classification error between biologically relevant and non-relevant tiles.

In this disclosure, the trained autoencoder may be obtained by training an autoencoder using an unsupervised approach, a semi-supervised approach, or a supervised learning approach.

In this disclosure, the trained CNN may be obtained by training a CNN using an unsupervised approach, a semi-supervised approach, or a supervised learning approach.

In this disclosure, the first set of training tiles and the second set of training tiles are created by an unsupervised approach, a semi-supervised approach, or a supervised learning approach.

Supervised learning may be a training approach that uses labeled data sets. For example, these labeled data sets may be designed to train or “supervise” an autoencoder or a CNN in classifying data or predicting outcomes accurately. Using labeled inputs and outputs, the autoencoder or the CNN may measure its accuracy and learn over time. Unsupervised learning, for example, may use machine learning algorithms to analyze and cluster unlabeled data sets. This approach may discover hidden patterns in data without the need for human intervention (hence, they may be applied “unsupervised”). Semi-supervised learning may be a learning approach combining supervised and unsupervised learning by using labeled and unlabeled data to train an autoencoder or a CNN for classification and regression tasks. Further examples of such learning and training approaches are disclosed at https://www.ibm.com/think/topics/supervised-vs-unsupervised-learning (accessed on Nov. 2, 2024.) and https://www.ibm.com/topics/semi-supervised-learning (accessed on Nov. 2, 2024.) The entire content of these references, including their supplemental content, is incorporated herein by reference.

In one example, the rare event identification system may be configured to use a (trained) autoencoder and/or a (trained) convolutional neural network (CNN) to identify biologically non-relevant events in a sample image. This configuration may include a step of obtaining a sample image segmented into multiple tiles, each tile containing data representing a portion of the sample image. This configuration may require training an autoencoder with all the tiles from this set in an unsupervised manner so that the autoencoder learns to reproduce these tiles. The autoencoder then may identify rare tiles from this set. A second step may involve counting the number of rare tiles within each frame of the original image and eliminating all rare tiles from a given frame if the number of rare tiles from that frame exceeds a threshold. This step may remove rare tiles that are associated with imaging artifacts. The third step may involve training a convolutional neural network (CNN) based classifier with a set of tiles that are identified as biologically relevant and another set identified as not relevant. This training may be performed in a supervised manner, wherein the labels associated with the tiles are also used to train the classifier. This classifier may then be applied to rare tiles to isolate biologically relevant ones.

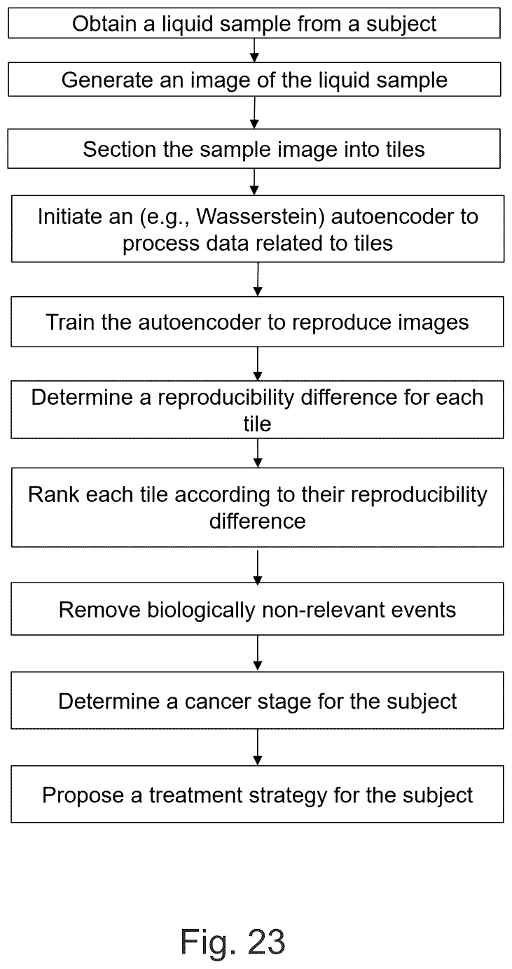

An exemplary method for determining a disease (e.g., cancer) state of a subject and/or proposal of a treatment strategy for the subject is illustrated in FIG. 23. To achieve these purposes, this method may use any rare event identification system of this disclosure. According to this method, a thin layer of a liquid sample is first formed on a flat substrate. Then, this liquid sample is stained using an immunofluorescence antibody. The stained liquid sample is scanned using an optical system of this disclosure. This scanning generates an image of the liquid sample. Then, this sample image is sectioned into a plurality of tiles. The rare event detection initiates an (e.g., Wasserstein) autoencoder to process data related to tiles; uses data related to a number or all tiles to train the autoencoder such that the autoencoder learns to reproduce each tile; determines a reproducibility difference for each tile; ranks each tile according to the reproducibility difference of each tile; and remove biologically non-relevant events. This method is further configured to determine a cancer state for the subject and propose a treatment strategy.

This invention disclosure describes a non-parametric approach for detecting biologically relevant rare events that circumvents some of the components of previous systems and methods, which are either time-consuming or require human intervention.

In this disclosure, the autoencoder may be any autoencoder. For example, the autoencoder may be a Wasserstein autoencoder (WAE), a denoising autoencoder, a sparse autoencoder, a deep autoencoder, a contractive autoencoder, an under-complete autoencoder, a convolutional autoencoder, a variational autoencoder, or a combination thereof. For example, the autoencoder may be a Wasserstein autoencoder (WAE).

In this disclosure, each tile may include a partial or complete image of at least one event. For example, the number of events in each tile may be in a range of 1 to 5, or a range of 1 to 10, or a range of 1 to 20, or a range of 1 to 30, or a range of 1 to 50, or a range of 1 to one 100, or a range of 1 to 1,000. For example, the number of events in each tile may be in a range of 1 to 5, or a range of 1 to 10.

In this disclosure, the sample image may be sectioned into at least 100 tiles, or at least 1,000 tiles, or at least 10,000 tiles, or at least 10,000,000 tiles, or at least 100,000,000 tiles. The maximum number of tiles to which the sample image is sectioned may be less than or equal to the total number of pixels of the sample image. For example, each tile may have a size of at least 100 pixels, or at least 1,000 pixels, or at least 10,000 pixels, or at least 50,000 pixels, or at least 100,000 pixels, or at least 1,000,000 pixels. For example, each tile may have a size of at least 100 pixels, or at least 1,000 pixels, or at least 10,000 pixels. For example, each tile may have a size in a range of 100 pixels to 1,000 pixels, or in a range of 100 pixels to 10,000 pixels, or a range of 100 pixels to 100,000 pixels, or in a range of 1,000 pixels to 10,000 pixels. For example, each tile may be of a size that contains at least one partial or complete image of at least one event. For example, each tile may have a size such that it can include at least one event but no more than 3, or no more than 5, or no more than 10, or no more than 30, or no more than 50, or no more than 100, or no more than 1,000 partial or complete images of events. For example, each tile may have a size such that it can contain at least one event but at most 3, or at most 5 partial or complete images of events.

In this disclosure, the reproducibility difference may be determined, for example, by using calculated values of a weighted vector norm, a weighted tensor norm, a vector norm, a tensor norm, or a combination thereof of the difference between the original tile's data and the reproduced tile's data; and wherein a calculated value of said norms or a combination thereof is hereafter referred to as rarity metric. For example, the reproducibility difference may be determined using calculated values of a vector norm or a tensor norm of the difference between the original tile's data and the reproduced tile's data.

For example, the reproducibility difference may be determined by using calculated values of a vector norm. Exemplary vector norms are disclosed, for example, by Wolfram MathWorld at https://mathworld.wolfram.com/VectorNorm.html (accessed on Mar. 16, 2024.) The entire content of this reference, including its supplemental content, is incorporated herein by reference. Following the notations of this reference, given an n-dimensional vector:

$$x = [ \begin{matrix}{x}_{1} \\ {x}_{2} \\ ⋮ \\ {x}_{n}\end{matrix} ] , \qquad Equation ( 1 )$$

Then, the vector norm [x] for p=1, 2, . . . is defined as:

$${\vert x \vert}_{p} ≡ {( {\sum }_{i} {\vert {x}_{i} \vert}^{p} )}^{1 / p} \qquad Equation ( 2 )$$

The special case, infinity vector norm, [x]_{∞} is defined as:

$${\vert x \vert}_{∞} ≡ {max}_{i} \vert {x}_{i} \vert . \qquad Equation ( 3 )$$

For example, L2-norm or L² norm is defined as:

$${\vert x \vert}_{2} = \vert x \vert = \sqrt{{x}_{1}^{2} + {x}_{2}^{2} + … + {x}_{n}^{2}} \qquad Equation ( 4 )$$

In the above equations, Equations (1)-(4), x is a difference between an input to the autoencoder and output from the autoencoder. The input to the autoencoder may be data related to a tile of the obtained (i.e., original) sample image, and the output from the autoencoder may be data related to a tile of the reproduced (i.e., synthetic) sample image. Then, for example, x is a difference between the intensity of a data point (e.g., of a pixel) on the obtained sample image's tile and the intensity of a data point (e.g., of a pixel) on the reproduced sample image's tile, wherein said obtained sample image's tile's data point and said reproduced sample image's tile's data point have same locations.

In this disclosure, the tiles may be ranked from most rare to least rare or normal, or least rare or normal to most rare, according to the rarity metric of each tile. A tile referred to as a rare tile may have a high rarity metric value. A tile that is referred to as a normal tile or a least rare tile may have a low rarity metric value. A tile referred to as a rarest tile may have the highest rarity metric value. A tile referred to as a most-normal tile may have the smallest rarity metric value. For example, a rare tile may be determined using a value of the calculated vector norm or tensor norm and a threshold rarity metric value for the computed norm's value or the calculated tensor's value. For example, the threshold rarity metric may be predetermined such that the rare event identification system may identify in a range of 1 to 10 rare tiles, or a range of 1 to 100 rare tiles, or a range of 1 to 1,000 rare tiles, or a range of 1 to 10,000 rare tiles, or a range of 1 to 100,000 rare tiles, which have rarity index values larger than those of remaining rare tiles and the normal tiles. For example, the threshold rarity metric may be predetermined such that the rare event identification system may identify in a range of 1 to 10 rare tiles, or a range of 1 to 100 rare tiles, or a range of 1 to 1,000 rare tiles, which have rarity index values larger than those of remaining rare tiles and the normal tiles.

In this disclosure, the rare event identification system is configured to obtain plurality of sample images or process data related to the plurality of tiles sectioned from a plurality of sample images. These sample images may be formed by using a fluorescence spectroscopy system and/or a bright field imaging system (e.g., a bright field microscope). The fluorescence spectroscopy system may have more than 1 channel, or more than 2 channels, or more than 3 channels, or more than 4 channels, or more than 5 channels, or more than 6 channels, or more than 7 channels, or more than 8 channels, or more than 9 channels, or more than 10 channels. Each fluorescence channel of such a fluorescence spectroscopy system may form a sample image at a fluorescence wavelength different than that of the other fluorescence channels. Such fluorescence spectroscopy system thereby allows interrogation and identification of one or a plurality of different biological structure(s), for example, through their biological markers (“markers”). For example, as disclosed above, a six-channel fluorescence spectroscopy system may be used to identify markers such as nucleus at about 390 nm (channel 1), BCMA at about 475 nm (channel 2), cytokeratin at about 555 nm (channel 3), ER at about 575 nm (channel 4), PR at about 635 nm (channel 5), and CD45 at about 748 nm (channel 6). One channel may be used in the identification of one or more than one biomarker. In addition, a bright field imaging system may also form a sample image. The rare event identification system(s) of this disclosure is configured to generate, use, obtain, and/or process data related to one or more sample images formed by using a one or multi-channel fluorescence spectroscopy system and/or a bright field imaging system. In one example of such rare event identification system(s) of this disclosure, the rare event identification system is configured to obtain a plurality of sample images, wherein: (a) each sample image of a group of sample images may be formed by using a different fluorescence channel dedicated to identifying a specific marker(s) associated with a biological structure; and (b) the sample image group comprises at least 2 sample images, or at least 3 sample images, or at least 4 sample images, or at least 5 sample images, or at least 6 sample images, or at least 7 sample images, or at least 8 sample images, or at least 9 sample images, or at least 10 sample images. In another example such rare event identification system(s) of this disclosure, the rare event identification system is configured to obtain a plurality of sample images, wherein: (a) at least one sample image is formed by using a bright-field imaging system; (b) each sample image of a group of sample images is formed by using a different fluorescence channel dedicated to identifying a specific marker(s) associated with a biological structure; and (c) the sample image group comprises at least 2 sample images, or at least 3 sample images, or at least 4 sample images, or at least 5 sample images, or at least 6 sample images, or at least 7 sample images, or at least 8 sample images, or at least 9 sample images, or at least 10 sample images. In a refinement, the system receives a plurality of pre-provided tiles that represent sectioned portions of a sample image. The system employs a trained autoencoder to process these tiles, with the autoencoder generating reproduced tile data for each tile. The system then calculates the reproducibility difference between each original tile's data and its corresponding reproduced tile's data, using these differences to rank the tiles accordingly. In a further refinement, the system operates by accepting pre-provided tiles representing sectioned portions of a sample image and processing them through a trained autoencoder without requiring image acquisition or sectioning operations. The system computes the reproducibility difference between the original tile's data and the corresponding reproduced tile's data for each tile, and subsequently ranks the tiles based on their respective reproducibility differences. In this disclosure, the rare event identification system may further be configured to determine the presence of a rare event in each tile.

This disclosure covers any combination of inventive features related to rare event identification systems and methods.

The following examples illustrate the various embodiments of the present invention. Those skilled in the art will recognize that many variations are within this disclosure's scope.

### EXAMPLE 1. Exemplary Materials and Methods

In this example, the sample images were generated by following materials and methods of the Setayesh publication, “Setayesh, S. M., Hart, O., Naghdloo, A., Higa, N., Nieva, J., Lu, J., and Kuhn, P. (2022). Multianalyte liquid biopsy to aid the diagnostic workup of breast cancer. NPJ Breast Cancer, 8 (1), 112.” The entire content of this publication, including its supplemental content, if available, is incorporated herein by reference. The following Setayesh materials and methods were briefly as follows.

### Study Design

A total of 100 BC patients and 30 normal donors are included in this study. Cancer patients were recruited to the prospective Physical Sciences in Oncology study (PSOC-0068) entitled OPTImization of blood COLLection (OPTICOLL). Here, we present a subset consisting of 74 patients clinically classified as early-stage and 26 patients clinically classified as late-stage BC at the time of enrollment (Table 1). All cancer patients were enrolled between April 2013 and Jan. 17, 2017, at multiple clinical sites in the United States: Billings Clinic (Billings, MT), Duke University Cancer Institute (Durham, NC), City of Hope Comprehensive Cancer Center (Duarte, CA), and University of Southern California Norris Comprehensive Cancer Center (Los Angeles, CA). Patient recruitment took place according to an institutional review board-approved protocol at each site, and all study participants provided written informed consent.

TABLE 1
Clinical demographics for Keck subset of patients.

| | | |
|---|---|---|
| Age | 71.4 | (53.4-86.1) |
| BMI | 24.9 | (21.2-36.9) |
| Hgb | 11.1 | (5.1-15.0) |
| HCT | 34.2 | (18.3-46.3) |
| WBC | 7.6 | (4.8-20.4) |
| Platelets | 201.5 | (57-387) |
| BUN | 22.5 | (13-70) |
| Creatinine | 1.2 | (0.5-3.1) |

| | | |
|---|---|---|
| Race | Caucasian | 20 |
| | Asian | 2 |
| Gender | Male | 18 |
| | Female | 4 |
| Smoker | Previous | 14 |
| | Current | 4 |
| | Never | 4 |
| Neoadjuvant Chemo | Yes | 10 |
| | No | 12 |
| Surgical Procedure | Anterior Exenteration | 1 |
| | Radical Cystectomy | 4 |
| | Robotic Radical | 17 |
| | Cystectomy | |
| Urinary Diversion | Studer | 9 |
| | Ileal Conduit | 11 |
| | Indiana Pouch | 2 |
| Pure Urothelial (CS/PS) | | 7/4 |
| Predominant Histology | No Tumor | 2/9 |
| (CS/PS) | Urothelial | 17/11 |
| | Other | 3/1 |
| | Plasmacytoid | 0/1 |
| Squamous (CS/PS) | Absent | 16/12 |
| | Present | 2/1 |
| | NA | 4/9 |
| Glandular (CS/PS) | Absent | 16/12 |
| | Present | 2/1 |
| | NA | 4/9 |
| Neuro (CS/PS) | Absent | 18/12 |
| | Present | 1/1 |
| | NA | 3/9 |
| Subgroup (CS/PS) | OC | 16/15 |
| | EV | 4/3 |
| | N+ | 2/4 |
| T Stage (CS/PS) | T0 | 2/9 |
| | Ta | 2/0 |
| | Tis | 1/4 |
| | T1 | 1/2 |
| | T2a | 11/0 |
| | T2b | 0/1 |
| | T3a | 0/3 |
| | T3b | 2/1 |
| | T4a | 3/2 |
| N Stage (CS/PS) | NX | 2/0 |
| | N0 | 19/18 |
| | N2 | 1/4 |

Abbreviations: CS: clinical staging; PS: pathological staging; OC: organ confined; EV: extravesical; N+: node positive; BMI: body mass index; Hgb: hemoglobin; HCT: hematocrit; WBC: white blood cell; BUN: blood urea nitrogen.

The study schedules were coordinated and unified across the clinical sites. For patients included in this study with non-metastatic treatment naïve disease (early-stage BC), the blood draws were acquired before any treatment. Patients with metastatic disease (late-stage BC) had multiple blood specimens collected at the beginning of a new line of therapy, either as a first line of therapy or post-progression while on therapy for the treatment of metastatic malignancy. A total of 10 normal blood donor samples were procured from the Scripps Clinic Normal Blood Donor Service and defined as individuals with no known pathology. Additionally, 20 age and gender-matched normal donor samples were provided from Epic Sciences and defined as women between 45-82 years (median was 57 years) with no known pathology. Normal donors will refer to the accumulation of both Scripps Clinic and Epic Sciences samples.

### Blood Collection and Processing

Approximately 8 mL peripheral blood was collected in 10-mL blood collection tubes (Cell-free DNA BCT, Streck) at the respective clinical site. Blood specimens were shipped to and processed at the Convergent Science Institute in Cancer (CSI-Cancer) at the University of Southern California within 24-48 hours of collection, as previously described. Upon receipt, all samples underwent red blood cell lysis, and the remaining nucleated cell population was plated in a monolayer on custom-made cell adhesive glass slides (Marienfeld, Lauda, Germany) at approximately 3 million cells per slide. The prepped slides were subsequently incubated in 7% BSA, dried, and stored at −80° C.

### Immunofluorescence Assay

Two slides from each patient, corresponding to approximately 6 million nucleated cells, were thawed and subsequently stained using IntelliPATH FLX™ autostainer (Biocare Medical LLC, Irvine, CA, USA) in batches of 50 slides (46 patient slides [2 slides per patient] and 4 control slides) as previously described (20, 34, 36). All steps were performed at room temperature. Cells were fixed with 2% neutral buffered formalin solution (VWR, San Dimas, CA) for 20 min, nonspecific binding sites were blocked with 10% goat serum (Millipore, Billerica, MA) for 20 min. Slides were subsequently incubated with 2.5 μg/mL of mouse anti-human CD31 monoclonal antibody (Ab) (clone: WM59, MCA1738A647, BioRad, Hercules, CA) preincubated with 100 ug/mL of goat anti-mouse IgG monoclonal Fab fragments (115-007-003, Jackson ImmunoResearch, West Grove, PA) for 4 hr. After incubation with CD31-Fabs, cells were permeabilized using 100% cold methanol for 5 min. Cells were then incubated with an Ab cocktail consisting of mouse anti-human pan-cytokeratin (PanCK) mAbs (clones: C-11, PCK-26, CY-90, KS-1A3, M20, A53-B/A2, C2562, Sigma, St. Louis, MO), mouse anti-human CK19 mAb (clone: RCK108, GA61561-2, Dako, Carpinteria, CA), mouse anti-human CD45 Alexa Fluor® 647 mAb (clone: F10-89-4, MCA87A647, AbD serotec, Raleigh, NC), and rabbit anti-human vimentin (VIM) mAb (clone: D21H3, 9854BC, Cell Signalling, Danvers, MA) for 2 hr. Slides were then incubated with Alexa Fluor® 555 goat anti-mouse IgG1 antibody (A21127, Invitrogen, Carlsbad, CA) and counterstained with 4′,6-diamidino-2-phenylindole (D1306, ThermoFisher, Waltham, MA) for 40 min. Slides were then mounted with an aqueous mounting media to preserve cellular integrity for further downstream analysis.

### Image Acquisition and Feature Extraction

After staining, the slides were imaged using automated high-throughput fluorescence scanning microscopy at 100× magnification, resulting in 2304 image frames per slide, as previously reported. Exposure times and gain for PanCK, VIM, CD45/CD31, and DAPI (DNA) channels were determined computationally by the scanner control software to normalize the background intensity levels across all slides.

### EXAMPLE 2. Detecting Rare Events by Using a Wasserstein Auto Encoder (WAE)

A rare event identification system is configured to identify rare events in a sample as follows. This sample may be a biological sample. This biological sample may be a sample obtained from a mammal. This mammal may be a human. This human may or may not have a disease. These rare events may be biologically relevant rare events. These biologically rare events may be formed by a disease inflicting the mammal.

First, an image of a sample is obtained. This image may be a digital image. This image may include processable information comprising pixels and intensities of these pixels. This image is sectioned into a plurality of tiles. For example, the size of each tile may be 32 pixels×32 pixels (e.g., 19 micrometers×19 micrometers).

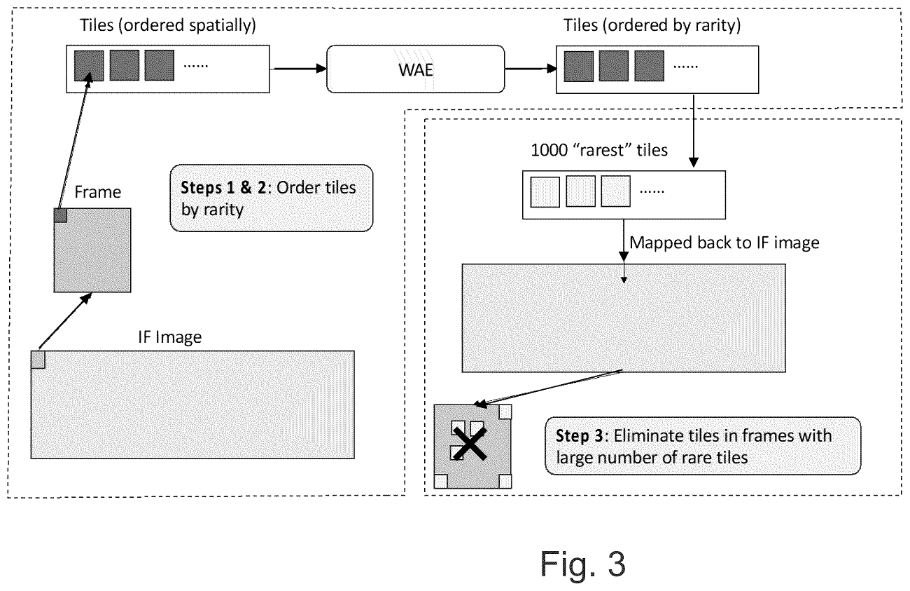

The rare event identification system is configured to process this information by running an autoencoder, for example, a Wasserstein Auto Encoder (WAE). The rare event identification system is configured to train WAE to learn a probability density function for all or some tiles of the sample image. The trained WAE generates a synthetic image of the plurality of tiles. The system may generate synthetic images of some or all tiles. Then, the rare event identification system determines the reproducibility difference between the true and synthetic tiles as a rarity metric for each tile. This rarity metric ranks the tiles, starting from the least rare tile to the rarest tile. Finally, the rare tiles that are not biologically relevant from this ranking are eliminated. This exemplary configuration of the rare event identification system is schematically shown in FIG. 3.

In one example, all tiles belonging to a sample image are used to train a WAE. During such an exemplary process, the WAE learns to reproduce these tiles. In another example, each tile is used as input to the WAE, which then reproduces the tile; that is, it forms a synthetic image of the tile.

After the training, the WAE learns to reproduce more common tiles, accurately. Thus, for these tiles, the reproducibility difference between the input to the WAE and the output (the reproduced or synthetic image) from the WAE is small. On the other hand, when rare tiles are used as input to the WAE, the autoencoder reproduces images of the tiles that are different from the input. Thus, the reproducibility difference between the input and the output tile image is significant in this case. We use this difference, for example, measured in the L² norm, as a measure or a metric of the rarity of a tile and are thus able to rank each tile in increasing order of rarity.

Immunofluorescence images typically have several immunofluorescence or fluorescence channels, where each channel is used to determine the intensity of a specific biomarker. Thus, when computing the L₂ distance described above, we have the freedom to weigh different channels differently. This choice determines each channel's relative role in determining an event's rarity. The higher the weight, the more significant the contribution from that channel to the rarity.

The rare event identification system may be further configured to apply two different approaches to remove tiles that are not biologically relevant. One or both approaches may be used to remove biologically non-relevant tiles, as follows.

In the first biologically non-relevant tile removal approach, the immunofluorescence image (i.e., the sample image), from which the tiles originate, is split into frames. For example, each frame may have a size of 1362 pixels×1004 pixels (803.58 micrometers×592.36 micrometers). The set of rare tiles is mapped back to their spatial location on their parent immunofluorescence image, and the number of rare tiles in each frame is enumerated. If the number of rare tiles in any given frame exceeds a threshold, then all those tiles are excluded from the set of rare tiles, considering that biologically relevant rare events are expected to be uniformly distributed in the entire immunofluorescence image. Hence, the probability of finding many such rare events near each other within a single frame is expectedly small. Therefore, if a large collection of such rare tiles is found within the same frame, these rare tiles are not biologically relevant; rather, such rare tiles' “rarity” may be attributed to errors in preparing the sample image. For example, the treatment of the sample before the imaging, which is used to prepare a sample for the imaging, or the imaging technique used to form the sample image may cause errors or noise that may lead to the formation of biologically non-relevant tiles on the sample image.

In the second biologically non-relevant tile removal approach, a deep learning-based classifier may be used to remove biologically non-relevant tiles. This deep learning-based classifier may be convolutional neural networks trained to classify tiles containing rare events but not biologically relevant. Such classifiers are trained by using a collection of tiles of two types. One collection set includes tiles with the known biologically not relevant event that is to be removed, and another collection set includes tiles of all other types. Once such classifier is trained, it is applied to the set of rare tiles to identify and remove the tiles that contain rare events but not biologically relevant.

### EXAMPLE 3. An Exemplary Architecture of a Wasserstein Autoencoder

An example of an autoencoder that rare event identification systems may use to process information related to tiles is a Wasserstein Autoencoder. An example of WAE is introduced by Tolstikhin et al. [1]. Like the variational auto-encoder (VAE), the goal of a WAE is to reconstruct the input by first mapping it to a latent vector of a much smaller dimension than the original input (this process is referred to as encoding the input) and then mapping the latent vector to a reconstruction of the image. However, WAE is different from VAE in how the latent space, i.e., the space of encoded data points, is regularized. In a WAE, the latent space is regularized by minimizing the Wasserstein distance between the latent space vectors and vectors sampled from a target prior distribution, which is typically chosen to be the standard normal distribution.

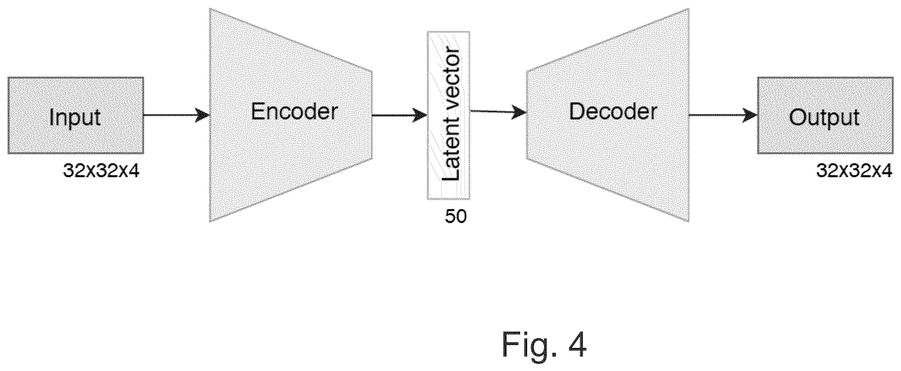

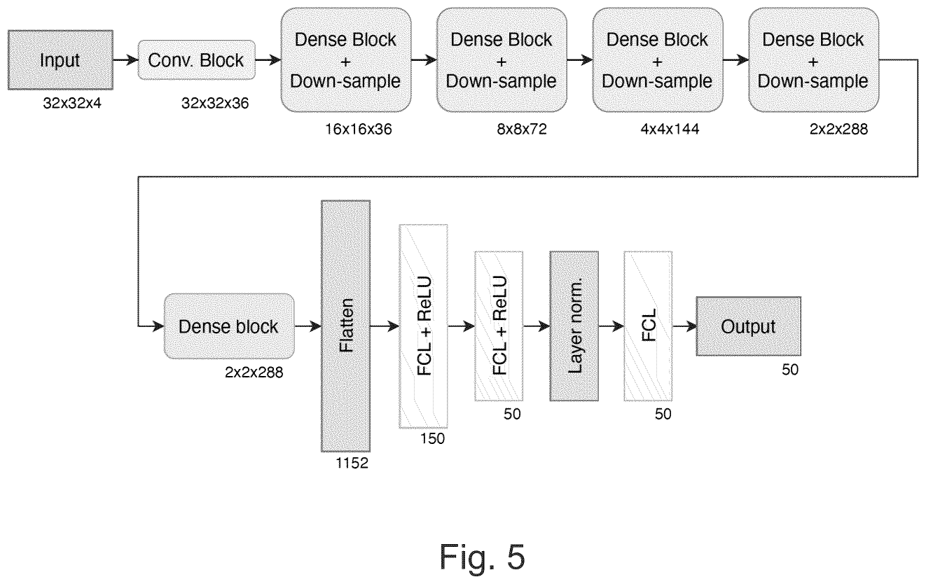

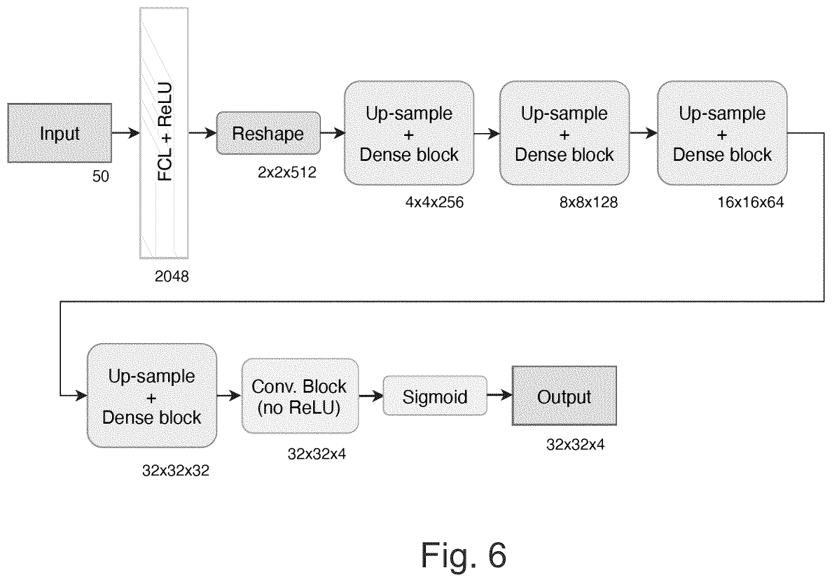

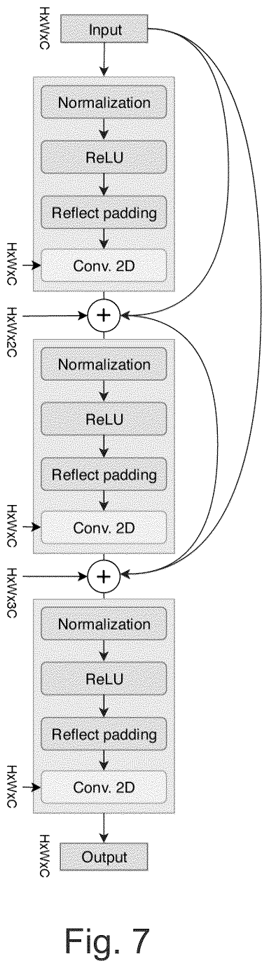

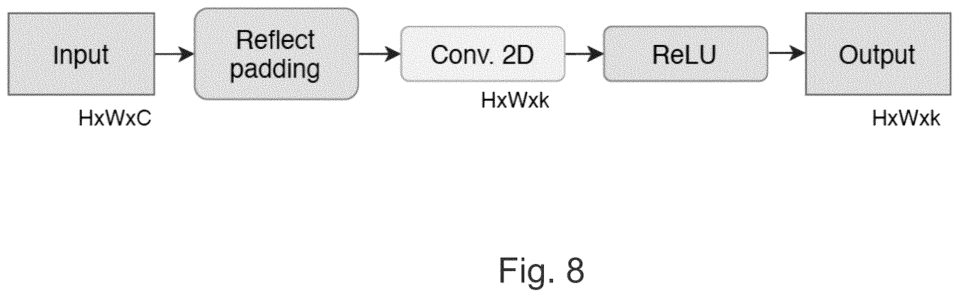

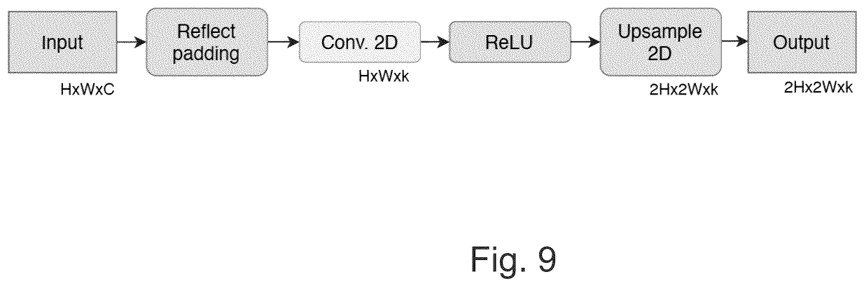

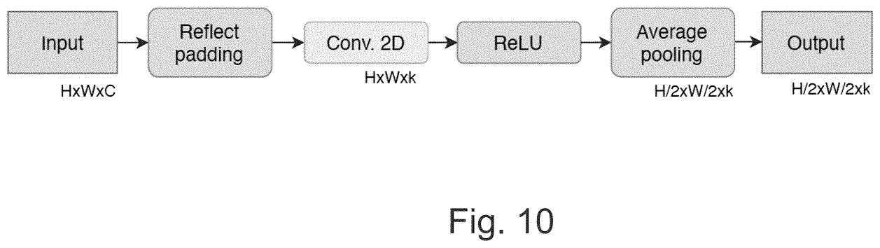

FIG. 4 shows an exemplary overall architecture of a WAE. FIGS. 5-6 show exemplary architectures that may be used for the encoder component and the decoder component (shown as blocks) of the WAE model. These encoder and decoder models may include a mixture of fully connected and convolutional layers. The main blocks used in these models are the convolution block, the dense block, the down-sample block, and the up-sample block. The dense block, shown in FIG. 7, may be based, for example, on the architecture introduced by Huang et al. [2]. FIGS. 8-10 show the convolution, the up-sample, and the down-sample architectures of a WAE, respectively.

### EXAMPLE 4. An Exemplary Loss Function

An exemplary loss function is disclosed in Equation 5. The rare event identification system is configured to minimize this loss function. This loss function may be a reconstruction loss given by the distance function c and a regularization of the encoded training data points so that they are distributed according to a target prior distribution, for example, a standard normal distribution.

$$ℒ ( {\{ {x}_{i} \}}_{i = 1}^{n} , {\{ {𝓏}_{i} \}}_{i = 1}^{n} , θ , ϕ ) = \frac{1}{n} {\sum }_{i = 1}^{n} c ( {x}_{i} , {G}_{θ} ( {𝒬}_{ϕ} ( {x}_{i} ) ) ) + {λ}_{z} M M D \qquad Equation ( 5 )$$

In the equation above {xᵢ}ᵢ₌₁ⁿ are the data samples, {zᵢ}ᵢ₌₁ⁿ are vectors from the target prior distribution, G_{θ} is the decoder, Q_{φ} is the encoder, θ is the vector of the decoder parameters, and φ is the vector of the encoder parameters.

An exemplary reconstruction error is defined in Equation 6 by the squared Euclidean distance. The distance function and the MMD loss are defined as

$$c ( x , {x}^{′} ) = {‖ x - {x}^{′} ‖}_{2}^{2} \qquad Equation ( 6 )$$

An exemplary latent space regularization is defined in Equation 7 using maximum mean discrepancy (MMD).

$$M M D = \frac{1}{n ( n - 1 )} {\sum }_{l \neq j}^{n} k ( {𝓏}_{l} , {𝓏}_{j} ) + \frac{1}{n ( n - 1 )} {\sum }_{l \neq j}^{n} k ( {𝒬}_{ϕ} ( {x}_{l} ) , {𝒬}_{ϕ} ( {x}_{j} ) ) + \frac{2}{{n}^{2}} {\sum }_{l j}^{n} k ( {𝓏}_{l} , {𝒬}_{ϕ} ( {x}_{j} ) ) \qquad Equation ( 7 )$$

Where n is the number of samples used to calculate the MMD and k is the kernel defined in Equation 4. In this example, we use the inverse multiquadric kernel following the implementation of Tolstikhin et al. [1]. The kernel used in the MMD term is given by

$$k ( 𝓏 , {𝓏}^{′} ) = \frac{2 {d}_{z} {σ}_{z}^{2}}{2 {d}_{z} {σ}_{z}^{2} + ‖ 𝓏 - {𝓏}^{′} ‖} \qquad Equation ( 8 )$$

Where d_{z} is the dimension of the latent space and σ₂ is the standard deviation of the assumed target Gaussian distribution.

### EXAMPLE 5. Exemplary Training Procedure

An exemplary procedure followed to train the encoder and decoder of a WAE is described in Table 2.

TABLE 2
Exemplary Training Procedure

```
Input: loss function [symbol]   , number of epochs N_{epochs}, encoder [symbol]   ,
decoder G_{θ}, training data set [symbol]   and batch size | [symbol]   |.
for i=1 to N_{epochs} do
  for j=1 to └| [symbol]   | / | [symbol]   |┘ do
    Sample [symbol]   = { [symbol]   ₁, [symbol]   ₂,..., [symbol]   } from [symbol]   without replacement.
    Sample [symbol]   = { [symbol]   ₁, [symbol]   ₂, ... , [symbol]   } from the prior P_{Z}.
    Sample [symbol]   = { [symbol]   ₁, [symbol]   ₂, ... , [symbol]   }, where [symbol]   ₁ = [symbol]   ( [symbol]   ₁).
    Upsample [symbol]   and G_{θ} by descending [symbol]   .
```

### EXAMPLE 6. Hyperparameters

Exemplary hyperparameters, that may be used in configuration of the rare event identification system and the values selected for these hyperparameters are shown in Table 3.

TABLE 3
Exemplary hyperparameters and their exemplary values.

| Hyperparameter | Value |
|---|---|
| Number of epochs, N_{epochs} | 50 |
| Learning rate, α | 10⁻⁵ |
| Latent space dimension, d_{z} | 50 |
| Activation function | ReLU |
| Batch size, \| [symbol] \| | 500 |
| Regularization parameter, λₚ | 10⁻⁷ |
| Latent space penalty, λ_{z} | 100 |
| Learning rate schedule | α * exp(−epoch/20) |

### EXAMPLE 7. Detection of Breast Cancer Related Cells and Vesicles

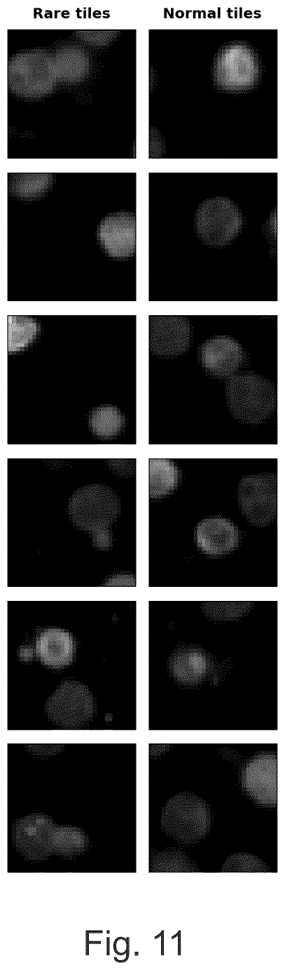

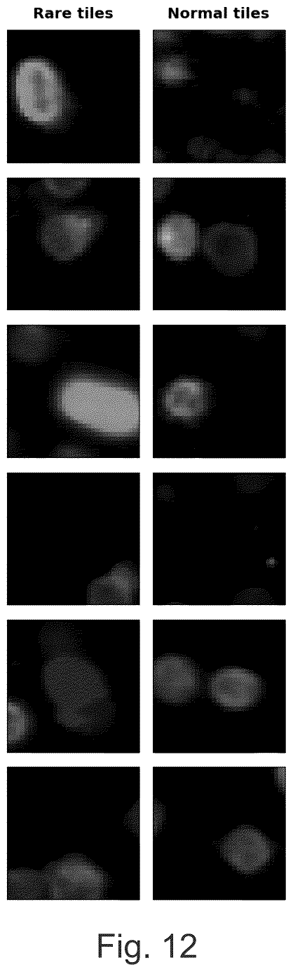

In this example, the rare event identification system was used to identify rare tiles on immunofluorescence images of a biological sample collected from a human subject with breast cancer. First, two datasets that include normal tiles and rare tiles were generated from this sample image, which included about 2.5 million tiles. Each dataset contained 180 tiles. Of these, 150 tiles were normal, and 30 tiles contained rare events, as identified by the rare event identification system. The 30 rare tiles were selected from or so tiles for the subject and either contained cancer-associated vesicles or circulating tumor cells. FIGS. 11-12 show six normal tiles and six rare tiles for each of the two datasets.

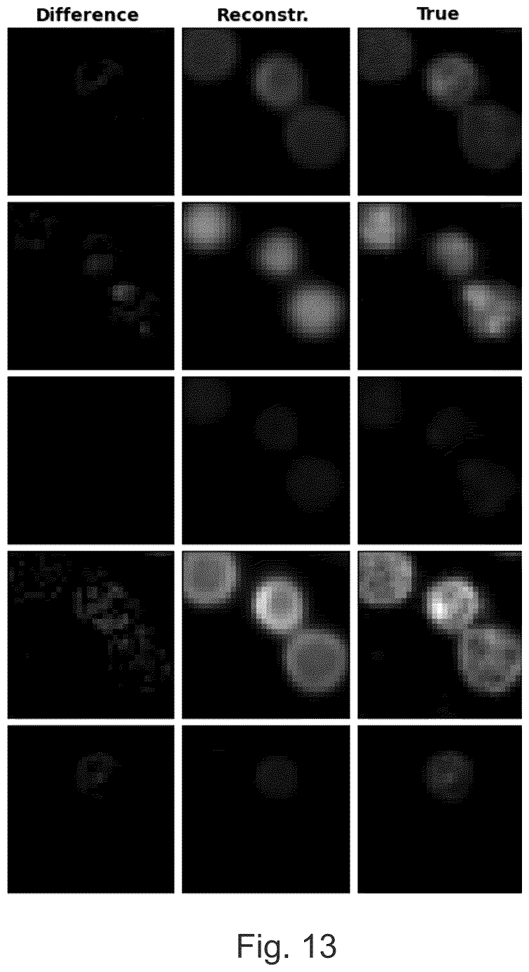

Each dataset was used to train a distinct WAE. Then, each tile (i.e., true image) was used as input to the trained WAE whose output was a reproduction (i.e., of reconstructed image) of the said tile. The reproducibility results are shown in FIGS. 13-16 for one representative normal tile and one representative rare tile for each of the two datasets (Dataset-1 and Dataset-2). In each case, the first row of tiles contains images of the true tile, the second row includes the reconstructed images, and the third row is the absolute value of the difference between the two. The first column is a composite color image of the four channels for the tile, and each subsequent column (there are 4) is a grayscale map of the intensity in each channel.

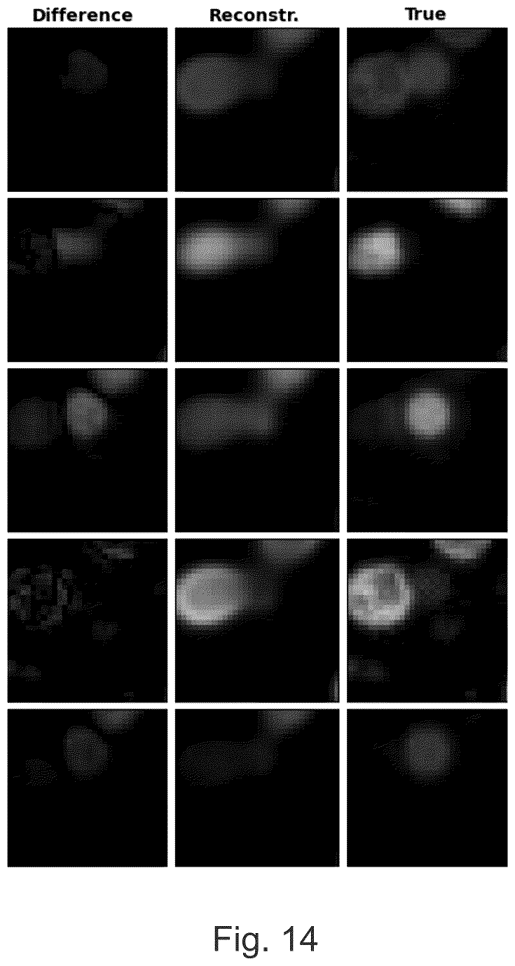

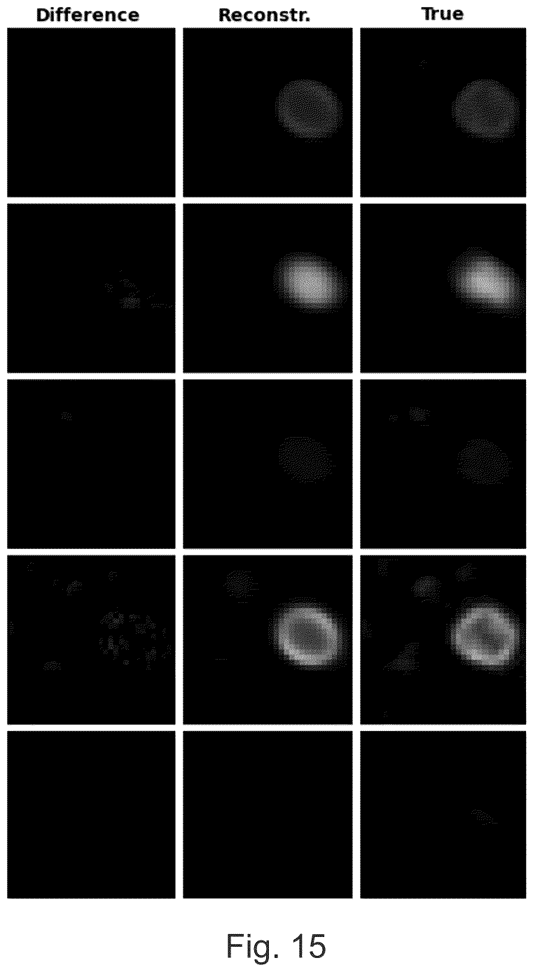

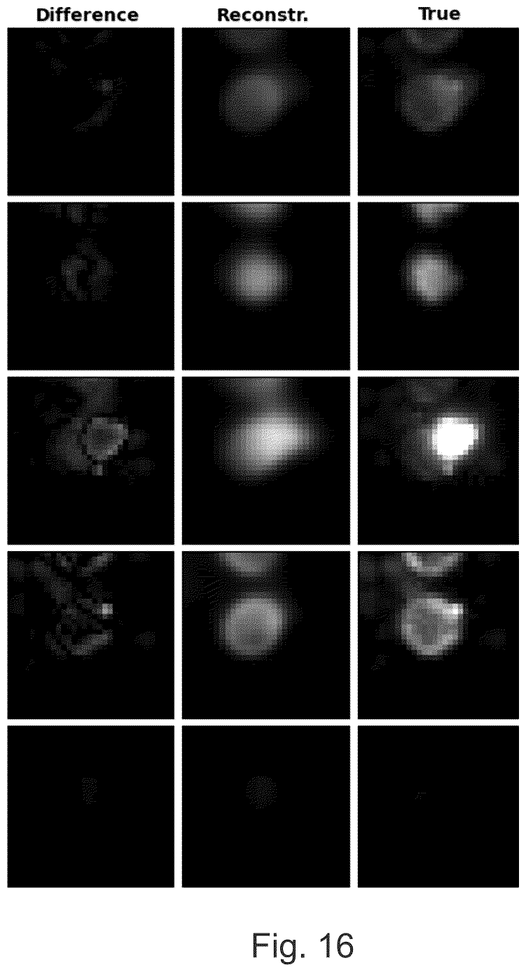

As shown in FIGS. 13 and 15, the rare event identification system reconstructed images of normal tiles substantially well, and, as such, the difference between the input images and the reproduced images was substantially small. See the “difference images” in said figures. In contrast, for the rare tiles, the reproduced tiles images were substantially different from the input (i.e., true) tile image. Consequently, the difference between the true image and the reconstructed image was large. See the “difference images” shown in FIGS. 14 and 16.

Next, the rare event identification system computes the L² norm of the difference image for each tile and uses this as a metric for the rarity of the tile. After that, rare event identification system determines the effectiveness of this metric in identifying the true rare tiles. The system accomplishes this by ranking the tiles in increasing order of their rarity metric and then considering different threshold values of this metric to classify the tiles as rare or normal.

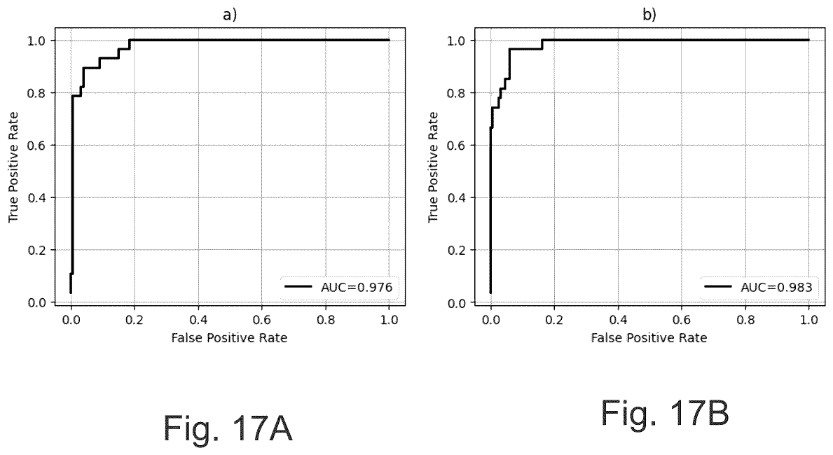

For the classification determined by each threshold, the rare event identification system computed the true positive and false positive rates. Also, by varying this threshold, the system generated a receiver operating characteristic (ROC) curve for the rarity metric (see FIG. 17). The area under this curve (AUC) was a quantitative measure of the performance of the rarity metric. An AUC=0.5 corresponded to a performance that was no better than random chance, whereas an AUC=1.0 indicated a perfect performance for the rarity metric. As such, the system achieved an AUC of 0.976 and 0.983, which determined that this disclosure's rare event identification system was highly effective in identifying rare tiles which can potentially be used in the cancer diagnosis.

### EXAMPLE 8. Detection of Breast Cancer Related Cells and Vesicles

For early-stage breast cancer patients, it has been shown that a single glass slide on which a liquid sample is deposited for immunofluorescence imaging may contain in a range of 2 to 300 vesicles. Such rare biological structures, e.g., vesicles, may indicate cancer. Given that each slide may contain around 6 million biological structures, such as cells in addition to vesicles, the occurrence of a vesicle in a tile may, therefore, be a rare event. Thus, we may quantify the efficacy of this disclosure's rare event identification system in detecting rare tiles via its ability to isolate vesicles in early-stage breast cancer patients.

In this example, the rare event identification system of this disclosure was used to identify rare events potentially present in biological samples obtained from 24 subjects with early-stage breast cancer. The number of tiles formed from an image of a single sample obtained from a subject was 2,531,840. The system of this disclosure processed these tiles. The system of the disclosure ranked these tiles from most rare to least rare.

Setayesh et al. [3] previously used another approach in the identification of the vesicles from the same immunofluorescence images of the samples obtained from these early-stage breast cancer patients. The number of vesicles identified by the Setayesh approach was treated as the “true” number of vesicles in the sample image in the evaluation of the effectiveness of the rare event identification system of this disclosure. The number of vesicles contained in the 500 most rare tiles was determined using this disclosure's system and compared with those of the approach disclosed in Setayesh, S. M., Hart, O., Naghdloo, A., Higa, N., Nieva, J., Lu, J., and Kuhn, P. (2022). Multianalyte liquid biopsy to aid the diagnostic workup of breast cancer. NPJ Breast Cancer, 8 (1), 112. The entire content of this publication, including its supplemental content, if available, is incorporated herein by reference. The results are summarized in Table 4.

TABLE 4
Effectiveness of the rare event identification
system in identifying vesicles.

| | | Vesicle count | | |
|---|---|---|---|---|
| The | Vesicle count, | by this | Vesicle count | |
| Sample | according to | disclosure's | by random tile | |
| Image ID | Setayesh | system | selection | Selectivity |
| 56778 | 262 | 58 | 0.066 | 885 |
| AB6602 | 189 | 56 | 0.047 | 1185 |
| AB6601 | 117 | 28 | 0.029 | 957 |
| 57794 | 113 | 35 | 0.028 | 1239 |
| 50748 | 97 | 39 | 0.024 | 1608 |
| 56264 | 68 | 13 | 0.017 | 765 |
| 57793 | 58 | 19 | 0.015 | 1310 |
| AFBF02 | 55 | 2 | 0.014 | 145 |
| 57374 | 46 | 12 | 0.012 | 1043 |
| 56263 | 41 | 14 | 0.010 | 1366 |
| 52684 | 41 | 24 | 0.010 | 2341 |
| 52683 | 39 | 19 | 0.010 | 1949 |
| ABF302 | 35 | 4 | 0.009 | 457 |
| 54689 | 35 | 17 | 0.009 | 1943 |
| AFAF02 | 31 | 7 | 0.008 | 903 |
| AC2001 | 28 | 2 | 0.007 | 286 |
| 56777 | 27 | 9 | 0.007 | 1333 |
| 74595 | 25 | 16 | 0.006 | 2560 |
| AC2002 | 25 | 5 | 0.006 | 800 |
| 59714 | 23 | 4 | 0.006 | 696 |
| 54688 | 22 | 10 | 0.006 | 1818 |
| 51474 | 21 | 11 | 0.005 | 2095 |
| AECF02 | 21 | 6 | 0.005 | 1143 |
| AC9901 | 21 | 3 | 0.005 | 571 |
| Average | 60 | 17 | 0.015 | 1225 |

Also presented in Table 4, the “vesicle count by random tile selection” column is the number of vesicles that would have been obtained in case the 500 tiles were selected at random and not ranked by rarity. As shown, the number of vesicles identified using the rare event identification system was significantly higher than those using the random tile selection approach. The average number of vesicles identified using this disclosure's system was 17, while the average value obtained from a random tile selection approach was 0.015.

The ratio of the number of vesicles identified by this disclosure's system to those identified by the random tile selection approach was a measure of the selectivity of this disclosure's system. This ratio, shown in column 5 of Table 4, was very large, with a mean of 1225, a minimum value of 145, and a maximum value of 2560. Thus, these results indicated that this disclosure's rare event identification system was approximately 1225 times more effective in identifying rare events of interest, such as vesicles.

In sum, the advantages of this disclosure's system in detecting outliers (e.g., rare events) in immunofluorescence microscopy images are as follows. The use of said system may:
(a) Eliminate the need to segment individual cells used in prior art approaches and replace them with a simple cropping operation, i.e., forming tiles and processing them to determine rare events.
(b) Eliminate the need to calculate cell image features from the segmented images of cells. This saves time.
(c) Reduce human intervention since the selection of which features are required to compute is typically done by a human expert.
(d) Provide a rarity ranking for tiles rather than just identifying which events are outliers.

Any combination of inventive features disclosed in the preceding paragraphs of this section or the paragraphs of the following sections is within the scope of this disclosure.

While exemplary embodiments are described above, these embodiments are not intended to represent all possible forms of the invention. Instead, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A rare event identification system configured to:
obtain an image of a sample;
section each sample image into a plurality of tiles;
initiate an autoencoder to process data related to the tiles;
use data related to a number or all tiles to train the autoencoder such that the autoencoder learns to reproduce each tile;
determine a reproducibility difference for each tile; and
rank each tile according to the reproducibility difference of each tile;
wherein:
the sample is derived from an object;
the image of the sample is hereafter referred to as the sample image;
the sample image comprises a plurality of pixels, and intensity of each of said pixels;
the tile is a sectioned image of the sample image;
the sample image is sectioned into a sufficient number of tiles to train the autoencoder;
a tile's data that is input to the autoencoder is hereafter referred to as an original tile's data;
the tile's data that is output from the autoencoder is hereafter referred to as a reproduced tile's data; and
the reproducibility difference is a difference between each original tile's data and a reproduced tile's data.

2. The rare event identification system of claim 1, comprising:
(a) an optical imaging system including: a liquid biopsy sample carrier suitable for supporting a liquid biopsy sample; an illumination system capable of illuminating the liquid biopsy sample at a specific wavelength or wavelengths that a fluorophore can absorb; a light detection system configured to detect and determine intensity and wavelength of fluorescence emitted by the fluorophore; and a light controlling system configured to allow detection of emitted, scattered, reflected, and/or transmitted electromagnetic radiation from the liquid biopsy sample and to guide electromagnetic radiation from the illumination system to the liquid biopsy sample and from the liquid biopsy sample to the light detection system, the light controlling system comprising at least one of an excitation filter, an emission filter, a (dichroic) mirror, a lens, and an optical fiber; and
(b) a processing system including a control system, a hardware processor, a memory system (28), and an information conveying system, wherein the hardware processor is configured to execute instructions stored in the memory system (28) to:
(i) receive image data from the light detection system (20) and generate a sample image of biological structure(s) present in the liquid biopsy sample;
(ii) section the sample image into a plurality of tiles;
(iii) initiate an autoencoder to process data related to the tiles and use data related to a number or all of the tiles to train the autoencoder such that the autoencoder learns to reproduce each tile;
(iv) for each tile, define original tile's data as input to the autoencoder and reproduced tile's data as output from the autoencoder, determine a reproducibility difference between the original tile's data and the reproduced tile's data, and rank the tiles according to their reproducibility differences; and
(v) responsive to identifying, within a region of the sample image comprising multiple tiles, a condition in which a proportion of tiles in the region have reproducibility differences that meet or exceed a predetermined criterion, command, via the control system (24), at least one of:
(v-a) the illumination system to reduce illumination intensity and/or to select a reduced subset of illumination wavelengths; and
(v-b) the light controlling system to select corresponding spectral channels by configuring at least one of the excitation filter and the emission filter, wherein the information conveying system is configured to communicate to a user information including locations of tiles ranked as rare on the sample image.

3. The rare event identification system of claim 1, wherein the autoencoder is a Wasserstein autoencoder (WAE), a denoising autoencoder, a sparse autoencoder, a deep autoencoder, a contractive autoencoder, an under-complete autoencoder, a convolutional autoencoder, a variational autoencoder, or a combination thereof.

4. The rare event identification system of claim 3, wherein the autoencoder is a Wasserstein autoencoder (WAE).

5. The rare event identification system of claim 1, wherein each tile comprises a partial or complete image of at least one event.

6. The rare event identification system of claim 5, wherein each tile has a size such that it can contain at least one partial or complete image of at least one event.

7. The rare event identification system of claim 1, wherein the reproducibility difference is determined by using calculated values of a weighted vector norm, a weighted tensor norm, a vector norm, a tensor norm, or a combination thereof of the difference between the original tile's data and the reproduced tile's data; and wherein a calculated value of these norms or a combination thereof is hereafter referred to as rarity metric.

8. The rare event identification system of claim 7, wherein the reproducibility difference is determined using calculated values of a vector norm or a tensor norm of the difference between the original tile's data and the reproduced tile's data.

9. The rare event identification system of claim 8, wherein the reproducibility difference is determined by using calculated values of a vector norm of the difference between the original tile's data and the reproduced tile's data, wherein the vector norm is an LP norm defined by Equation (2), or an L^{∞} norm defined by Equation (3), or a combination thereof.

10. The rare event identification system of claim 9, wherein the reproducibility difference is determined using calculated values, a vector norm, or a tensor norm, wherein the vector norm is an L¹ norm, an L² norm, or a combination thereof.

11. The rare event identification system of claim 10, wherein:
the tiles are ranked from most rare to least rare or normal, or least rare or normal to most rare, according to a rarity metric of each tile;
a tile that is referred to as a rare tile has a high rarity metric value;
a tile that is referred to as a normal tile or a least rare tile has a low rarity metric value;
a tile referred to as a rarest tile has a highest rarity metric value; and
a tile referred to as a most normal tile has a smallest rarity metric value.

12. The rare event identification system of claim 11, wherein the rare event identification system is further configured to determine a rare tile by using a value of a calculated vector norm or tensor norm and a threshold rarity metric value for a computed norm's value or a calculated tensor's value.

13. The rare event identification system of claim 12, wherein the rare event identification system is configured to identify in a range of 1 to 10 rare tiles, or a range of 1 to 100 rare tiles, or a range of 1 to 1,000 rare tiles, or a range of 1 to 10,000 rare tiles, or a range of 1 to 100,000 rare tiles, which have rarity index values larger than those of remaining rare tiles and normal tiles.

14. The rare event identification system of claim 13, wherein the rare event identification system is configured to identify in a range of 1 to 10 rare tiles, or a range of 1 to 100 rare tiles, or a range of 1 to 1,000 rare tiles, which have rarity index values larger than those of remaining rare tiles and normal tiles.

15. The rare event identification system of claim 12, wherein the rare event identification system is further configured to determine the presence of a rare event in each tile.

16. The rare event identification system of claim 15, wherein the rare event identification system is further configured to identify rare tiles that are not relevant to the object from which the sample is obtained; wherein non-relevant tiles include events that are erroneously introduced into the sample or the sample image or a tile image during the obtaining or processing of the sample image or the tile image; and wherein the said non-relevant tiles are hereafter referred to as biologically non-relevant tiles.

17. The rare event identification system of claim 16, wherein the rare event identification system is further configured to remove biologically non-relevant tiles from the sample image by:
sectioning the sample image into a plurality of image frames, wherein a size of each image frame is larger than that of a tile;
mapping back each rare tile back to its spatial location on the sample image;
counting the number of rare tiles on each image frame;
removing rare tiles from said image frame if a number of said rare tiles present in one image frame exceeds a threshold rare tile number; and
the threshold rare tile number is at least ten rare tiles, or 100 rare tiles, or 1,000 rare tiles in one image frame said tiles belong.

18. The rare event identification system of claim 1, wherein:
the rare event identification system is configured to obtain a plurality of sample images;
each sample image of a group of sample images is formed by using a different fluorescence channel dedicated to identifying a specific marker(s) associated with a biological structure; and
a sample image group comprises at least 2 sample images, or at least 3 sample images, or at least 4 sample images, or at least 5 sample images, or at least 6 sample images, or at least 7 sample images, or at least 8 sample images, or at least 9 sample images, or at least 10 sample images.

19. The rare event identification system of claim 18, wherein:
the rare event identification system is configured to obtain a plurality of sample images;
at least one sample image is formed by using a bright-field imaging system;
each sample image of a group of sample images is formed by using a different fluorescence channel dedicated to identifying a specific marker(s) associated with a biological structure; and
the sample image group comprises at least 2 sample images, or at least 3 sample images, or at least 4 sample images, or at least 5 sample images, or at least 6 sample images, or at least 7 sample images, or at least 8 sample images, or at least 9 sample images, or at least 10 sample images.

20. The rare event identification system of claim 16, wherein the rare event identification system is further configured to remove biologically non-relevant tiles from the sample image by using a trained convolutional neural network (CNN), wherein the trained convolutional neural network is trained on two sets of training tiles; wherein tiles belonging to a first training tile set comprises images of biologically non-relevant events; and wherein tiles belonging to a second training tile set includes only images of relevant events.

21. The rare event identification system of claim 20, wherein the rare event identification system is further configured to remove biologically non-relevant tiles from the sample image by using a trained convolutional neural network (CNN), wherein a CNN is trained by:
obtaining a sample image sectioned into a plurality of tiles, each tile containing data representing a portion of the sample image;
creating a first set of training tiles comprising images of biologically non-relevant events;
creating a second set of training tiles comprising images of biologically relevant events;
training the CNN on the first and second sets of training tiles to classify each tile as either biologically relevant or biologically non-relevant based on its learned features; and
thereby obtaining a trained CNN.

22. The rare event identification system of claim 21, wherein the CNN is trained to classify tiles based on features extracted from intensity values across multiple channels of the sample image, wherein each channel is associated with a specific biomarker.

23. The rare event identification system of claim 22, wherein the rare event identification system may be further configured to apply the trained CNN to a new set of tiles obtained from a sample image and to remove tiles classified as biologically non-relevant, thereby isolating tiles with biologically relevant events.

24. The rare event identification system of claim 23, wherein the rare event identification system is further configured to:
train a convolutional neural network (CNN) on a first set of training tiles and a second set of training tiles to obtain a trained CNN, wherein the first set of training tiles comprises tiles with biologically non-relevant events; and wherein the second set of training tiles comprises tiles with biologically relevant events; and
apply the trained CNN to remove tiles classified as biologically non-relevant, thereby isolating tiles with biologically relevant events for further analysis.

25. The rare event identification system of claim 24, wherein the CNN is trained with a supervised learning approach using a loss function that minimizes classification error between biologically relevant and non-relevant tiles.

26. A rare event identification system configured to:
initiate an autoencoder to process data related to a plurality of tiles;
use data related to a number or all tiles to train the autoencoder such that the autoencoder learns to reproduce each tile;
determine a reproducibility difference for each tile; and
rank each tile according to the reproducibility difference of each tile;
wherein:
the plurality of tiles are formed by sectioning an image of a sample;
the sample is derived from an object;
the image of the sample is hereafter referred to as a sample image;
the sample image comprises a plurality of pixels, and intensity of each of said pixels;
the tile is a sectioned image of the sample image;
the sample image is sectioned into a sufficient number of tiles to train the autoencoder;
a tile's data that is input to the autoencoder is hereafter referred to as an original tile's data;
the tile's data that is output from the autoencoder is hereafter referred to as a reproduced tile's data; and
the reproducibility difference is a difference between each original tile's data and a reproduced tile's data.

27. The rare event identification system of claim 1 comprising:
(a) an optical imaging system including a liquid biopsy sample carrier suitable for supporting a liquid biopsy sample, an illumination system capable of illuminating the liquid biopsy sample at a specific wavelength or wavelengths that a fluorophore can absorb, a light detection system configured to detect and determine intensity and wavelength of fluorescence emitted by the fluorophore, and a light controlling system, the light controlling system comprising at least one of an excitation filter, an emission filter, a (dichroic) mirror, a lens, and an optical fiber; and
(b) a processing system including a control system, a hardware processor, a memory system, and an information conveying system, wherein the hardware processor is configured to execute instructions stored in the memory system to:
(i) receive image data from the light detection system and generate a sample image of biological structure(s) present in the liquid biopsy sample;
(ii) section the sample image into a plurality of tiles, the plurality of tiles being formed by sectioning the sample image into a sufficient number of tiles to train an autoencoder;
(iii) initiate an autoencoder to process data related to the tiles and use data related to a number or all of the tiles to train the autoencoder such that the autoencoder learns to reproduce each tile;
(iv) for each tile, define original tile's data as input to the autoencoder and reproduced tile's data as output from the autoencoder, determine a reproducibility difference between the original tile's data and the reproduced tile's data, and rank the tiles according to their reproducibility differences; and
(v) responsive to identifying, during training, within a region of the sample image comprising multiple tiles, that a proportion of tiles have reproducibility differences meeting or exceeding a predetermined training rarity criterion, command, via the control system, at least one of:
(v-a) the illumination system (18) to select a reduced subset of illumination wavelengths for subsequent acquisition of that region; and
(v-b) the light controlling system to select corresponding spectral channels by configuring at least one of the excitation filter and the emission filter for subsequent acquisition of that region, wherein the information conveying system is configured to communicate to a user information including locations of tiles ranked as rare on the sample image.

28. The rare event identification system of claim 26, wherein the autoencoder is a Wasserstein autoencoder (WAE), a denoising autoencoder, a sparse autoencoder, a deep autoencoder, a contractive autoencoder, an under-complete autoencoder, a convolutional autoencoder, a variational autoencoder, or a combination thereof.

29. The rare event identification system of claim 28, wherein the autoencoder is a Wasserstein autoencoder (WAE).

30. The rare event identification system of claim 26, wherein each tile comprises a partial or complete image of at least one event.

31. The rare event identification system of claim 30, wherein each tile comprises a partial or complete image of a number of events and wherein the number of events is in a range of 1 to 5, or a range of 1 to 10.

32. The rare event identification system of claim 26, wherein the reproducibility difference is determined by using calculated values of a weighted vector norm, a weighted tensor norm, a vector norm, a tensor norm, or a combination thereof of the difference between the original tile's data and the reproduced tile's data, and wherein a calculated value of these norms or a combination thereof is referred to as a rarity metric.

33. A rare event identification system configured to:
obtain an image of a sample;
section each sample image into a plurality of tiles;
provide a trained autoencoder;
initiate the trained autoencoder to process data related to the tiles;
determine a reproducibility difference for each tile; and
rank each tile according to the reproducibility difference of each tile;
wherein:
the sample is derived from an object;
the image of the sample is hereafter referred to as the sample image;
the sample image comprises a plurality of pixels, and intensity of each of said pixels;
the tile is a sectioned image of the sample image;
a tile's data that is input to the trained autoencoder is hereafter referred to as an original tile's data;
the tile's data that is output from the trained autoencoder is hereafter referred to as a reproduced tile's data; and
the reproducibility difference is a difference between each original tile's data and a reproduced tile's data.

34. The rare event identification system of claim 33 comprising:
(a) an optical imaging system including a liquid biopsy sample carrier, an illumination system capable of illuminating a liquid biopsy sample at a specific wavelength or wavelengths that a fluorophore can absorb, a light detection system configured to detect and determine intensity and wavelength of fluorescence emitted by the fluorophore, and a light controlling system, the light controlling system comprising at least one of an excitation filter, an emission filter, a (dichroic) mirror, a lens, and an optical fiber; and
(b) a processing system including a control system, a hardware processor, a memory system, and an information conveying system, wherein the hardware processor is configured to execute instructions stored in the memory system to:
(i) receive image data from the light detection system and generate a sample image of biological structure(s) present in the liquid biopsy sample;
(ii) section the sample image into a plurality of tiles;
(iii) provide a trained autoencoder and initiate the trained autoencoder to process data related to the tiles;
(iv) for each tile, define original tile's data as input to the trained autoencoder and reproduced tile's data as output from the trained autoencoder, determine a reproducibility difference between the original tile's data and the reproduced tile's data, and rank the tiles according to their reproducibility differences; and
(v) responsive to identifying, within a region of the sample image comprising multiple tiles, that a proportion of tiles have reproducibility differences meeting or exceeding a predetermined rarity criterion, command, via the control system (24), at least one of:
(v-a) the illumination system to reduce illumination intensity and/or to select a reduced subset of illumination wavelengths; and
(v-b) the light controlling system to select corresponding spectral channels by configuring at least one of the excitation filter and the emission filter, wherein the information conveying system is configured to communicate to a user information including locations of tiles ranked as rare on the sample image.

35. The rare event identification system of claim 33, wherein the trained autoencoder is a Wasserstein autoencoder (WAE), a denoising autoencoder, a sparse autoencoder, a deep autoencoder, a contractive autoencoder, an under-complete autoencoder, a convolutional autoencoder, a variational autoencoder, or a combination thereof.

36. The rare event identification system of claim 35, wherein the trained autoencoder is a Wasserstein autoencoder (WAE).

37. The rare event identification system of claim 33, wherein the reproducibility difference is determined by using calculated values of a weighted vector norm, a weighted tensor norm, a vector norm, a tensor norm, or a combination thereof of the difference between the original tile's data and the reproduced tile's data, and wherein a calculated value of these norms or a combination thereof is referred to as a rarity metric.

38. The rare event identification system of claim 37, wherein the tiles are ranked from most rare to least rare or normal according to a rarity metric of each tile; a tile referred to as a rare tile has a high rarity metric value; a tile referred to as a normal tile has a low rarity metric value; a tile referred to as a rarest tile has a highest rarity metric value; and a tile referred to as a most-normal tile has a smallest rarity metric value.

39. The rare event identification system of claim 38, wherein the rare event identification system is further configured to determine a rare tile by using a value of a calculated vector norm or tensor norm and a threshold rarity metric value for a computed norm's value or a calculated tensor's value.

40. The rare event identification system of claim 33, wherein the system is configured to receive a plurality of pre-provided tiles representing sectioned portions of a sample image, and to process said tiles using a trained autoencoder, wherein the trained autoencoder reproduces each tile to generate reproduced tile data, and the system determines a reproducibility difference between each original tile's data and the corresponding reproduced tile's data, and ranks the tiles according to the reproducibility difference of each tile.

41. The rare event identification system of claim 33, wherein the system is configured to receive a plurality of pre-provided tiles representing sectioned portions of a sample image, and to process the pre-provided tiles using a trained autoencoder, wherein the system processes the pre-provided tiles without performing image acquisition or sectioning operations, and is configured to determine a reproducibility difference between each original tile's data and a corresponding reproduced tile's data, and to rank the tiles according to the reproducibility difference of each tile.
